(19) United States
(12) Patent Application Publication
(10) Pub. No.: US 2025/0177377 A1
(43) Pub. Date: Jun. 5, 2025

(54) TARGETING PTCHD1 AND NEURONAL CHOLESTEROL TO ENHANCE SAFETY OF OPIOID ANALGESICS

(71) Applicant: University of Florida Research Foundation, Incorporated, Gainesville, FL (US)

(72) Inventors: Kirill MARTEMYANOV, Gainesville, FL (US); Brock GRILL, Gainesville, FL (US)

(21) Appl. No.: 18/842,120

(22) PCT Filed: Mar. 9, 2023
(86) PCT No.: PCT/US2023/064020

§ 371 Date: Aug. 28, 2024

Related U.S. Application Data
(60) Provisional application No. 63/319,113, filed on Mar. 11, 2022.

(51) Int. Cl. A61K 31/485; A61K 31/192; A61K 31/20; A61K 31/216; A61K 31/337; A61K 31/352; A61K 31/395; A61K 31/505; A61K 31/785; A61K 39/395; A61K 49/00; C12N 9/22; C12N 15/11; C12N 15/113; C12N 15/90

(52) U.S. Cl. CPC A61K 31/485; A61K 31/192; A61K 31/20; A61K 31/216; A61K 31/337; A61K 31/352; A61K 31/395; A61K 31/505; A61K 31/785; A61K 39/3955; A61K 49/0008; C12N 9/22; C12N 15/11; C12N 15/113; C12N 15/907; C12N 2310/11; C12N 2310/14; C12N 2310/20

(57) ABSTRACT

Described are compositions and methods for increasing opioid therapy efficacy and ameliorating opioid use disorder. The compositions and methods target Ptchd1 or neuronal cholesterol.

### CROSS-REFERENCE TO RELATED APPLICATIONS

The application claims the benefit of U.S. Provisional Application No. 63/319,113, filed Mar. 11, 2022, which is incorporated herein by reference.

### STATEMENT REGARDING FEDERALLY SPONSORED RESEARCH OR DEVELOPMENT

This invention was made with government support under grant numbers, R21 DA040406 and R01 DA048036, R01 DA036596 and R01 EY028033, F32 DA047771, awarded by the National Institutes of Health. The government has certain rights in the invention.

### SEQUENCE LISTING

The Sequence Listing written in file 589806_SeqListing_ST26.xml is 14 kilobytes in size, was created Feb. 17, 2023, and is hereby incorporated by reference

### BACKGROUND

G protein Coupled Receptors (GPCRs) comprise the largest family of cellular receptors that mount responses to a large range of stimuli in eukaryotes. Studies over the past two decades have shown that GPCR signaling involves transducers, adaptors, and scaffolding proteins that control receptor trafficking and function. However, the molecular and cellular mechanisms underlying several key GPCR properties remain poorly understood. One such poorly understood area is tolerance, a behavioral phenomenon that ensues from the cellular process of GPCR desensitization. The ability to attenuate responses following stimulation is a fundamental feature of many GPCRs and is thought to be an adaptive physiological mechanism that prevents signaling overflow. However, this desensitization poses limitations in pharmacologically targeting GPCRs and developing effective therapeutics. A quintessential example of this problem is opioid tolerance. Opioid tolerance results in a significant loss of analgesic and euphoric efficacy upon chronic drug exposure, leading to escalation of use, addiction, and overdose.

The clinically significant effects of opioids are mediated by their target GPCR, the μ-opioid receptor (MOR). Previous studies have shown that alterations in MOR signaling and trafficking impact the development of tolerance in a cell autonomous manner. A few disparate molecules have been implicated in opioid tolerance, included β-arrestin. However, the molecular landscape of players and processes that GPCRs utilize to diminish their responses upon persistent stimulation remains incompletely defined. Identification of such players could be used to develop therapeutic approaches to overcome opioid tolerance, treat addiction, or treat withdrawal.

### SUMMARY

Described are Ptched1 and/or Ptchd1 pathway antagonists, pharmaceutical compositions containing the Ptchd1 or Pichd1 pathway antagonists, and methods of using the antagonists and pharmaceutical compositions to modulate opioid tolerance and efficacy.

Described are methods for inhibiting or reducing Ptchd1 expression or activity or Ptchd1 pathway activity. The methods comprise administering to a subject a therapeutically effective amount of any one or more Ptchd1 or ptchd1 pathway antagonists. Administering a therapeutically effective amount of a Ptchd1 or ptchd1 pathway antagonist includes, but is not limited to, administering to the subject a pharmaceutical composition that contains the therapeutically effective amount of the Ptchd1 or Ptchd1 pathway antagonist. A Ptchd1 antagonist or a Ptchd1 pathway antagonist can be used to enhance an analgesic effect mediated by the μ-opioid receptor (MOR) in a subject. Inhibiting Ptch1 expression or activity or inhibiting the Ptchd1 pathway can lead to stimulation or enhancement of MOR mediated analgesic effect in the subject. Inhibiting Ptch1 expression or activity or inhibiting Ptchd1 pathway activity can be used to reduce opioid tolerance. Inhibiting Ptch1 expression or activity or inhibiting Ptchd1 pathway activity is also expected to ameliorate opioid withdrawal and thus be beneficial for combating dependence by facilitating opioid abstinence.

In some embodiments, a subject is administered with an opioid drug for pain relief and one or more Ptchd1 or Ptchd1 pathway antagonists, wherein the one or more Ptchd1 or Ptchd1 pathway antagonists enhances the effect of the opioid drug. The opioid drug can be administered to the subject prior to, simultaneously with, or subsequent to administration of the one or more Ptchd1 or Ptchd1 pathway antagonists. In some embodiments, the opioid drug and the Ptchd1 or Ptchd1 pathway antagonists are formulated together. In some embodiments, the opioid drug and the Ptchd1 or Ptchd1 pathway antagonists are formulated for separate administration. The opioid drug can be, but is not limited to, oxycodone, hydrocodone, morphine, codeine, dihydrocodeine, fentanyl, buprenorphine, or methadone. The subject can be, but is not limited to, a human.

In some embodiments, methods are described for suppressing or ameliorating withdrawal symptoms in subjects suffering from chronic use of an opioid drug (e.g., addiction), to assist a subject in reducing dependence on an opioid drug, to assist a subject in reducing opioid use, or to treat an opioid use disorder. These methods comprise administering to the subject an effective amount of one or more Ptchd1 or Pichd1 pathway antagonists to suppress or ameliorate one or more withdrawal symptoms in the subject. The subject can be suffering from one or more opioid withdrawal symptoms or at risk of suffering from one or more opioid withdrawal symptoms. In some methods, a Ptchd1 or Ptchd1 pathway antagonist is administered to a subject after discontinuing or reducing use of the opioid drug. In some methods, a Ptchd1 or Ptchd1 pathway antagonists is administered to a subject prior to discontinuing or reducing use of the opioid drug. Reducing use of the opioid drug includes, but is not limited to, reducing dosage or frequency of administration of the opioid drug. The opioid drug can be, but is not limited to, oxycodone, hydrocodone, morphine, codeine, dihydrocodeine, heroin, opium, or fentanyl. The subject can be, but is not limited to, a human.

In some embodiments, a Ptchd1 antagonist comprises a nucleic acid-based inhibitor. In some embodiments, a nucleic acid-based inhibitor comprises an RNA interference (RNAi) polynucleotide or an antisense oligonucleotide (ASO). An RNAi polynucleotide can be, but is not limited to, an siRNA, an shRNA or a nucleic acid encoding an siRNA or shRNA. Contacting a cell with, or introducing into the cell, an RNAi polynucleotide or an antisense oligonucleotide inhibits expression of the Ptch1 gene in the cell. Inhibiting expression of the Ptch1 gene leads to decreased Ptchd1 activity in the cell. In some embodiments, a nucleic acid-based inhibitor comprises a CRISPR-based system.

Ptch1 activity leads to decreased cholesterol in neural tissue. In some embodiments, inhibiting Ptch1 pathway activity comprises increasing cholesterol in neuronal tissue. Agents that increase cholesterol in neuronal tissue include, but are not limited to, statins, cholesterol absorption inhibitors, PCSK9 inhibitors, citrate lyase inhibitors, bile acid sequestrants, HDL-C raising therapeutics, fibrates, niacin, omega-3 fatty acids, APOE-based therapeutics, and ABCA1 cholesterol transporter-based therapeutics, Statins include, but are not limited to, atorvastatin, fluvastatin, lovastatin, pitavastatin, pravastatin, rosuvastatin, and simvastatin, Cholesterol absorption inhibitors include, but are not limited to, ezetimibe, PCSK9 inhibitors include, but are not limited to, alirocumab and evolocumab, Citrate lyase inhibitors include, but are not limited to, bempedoic acid and bempedoic acid-ezetimibe, Bile acid sequestrants include, but are not limited to, cholestyramine, colesevelam and colestipol, HDL-C raising therapeutics include, but are not limited to, rosuvastatin, simvastatin, and atorvastatin. Fibrates include, but are not limited to, fenofibrate and gemfibrozil. Omega-3 fatty acids include, but are not limited to, Jovaza, omacor, and vascepa, APOE-based therapeutics include, but are not limited to, CN-105, CS-6253, a phthalazinone, a pyrazoline, an anti-APOE antibody, an APOE4 ASO (antisense oligonucleotide), an APOE4 RNAi polynucleotide, bexarotene, probucol, AGB101, and epigallocatechin gallate. ABCA1 cholesterol transporter-based therapeutics include, but are not limited to, probucol.

In some embodiments, the Ptchd1 pathway is inhibited using naturally occurring ligands of the Ptch system. Such ligands include, but are not limited to Sonic hedgehog (Shh), which blocks the Ptch/Ptchd1 pathway.

In some embodiments, the Ptchd1 pathway is inhibited using commercially available drugs that block interaction of Ptchd pathway components. Such drugs include, but are not limited to cyclopamine, which blocks Ptch/Shh interaction, vismodegib, and sonidegib.

Also described are modified C. elegans animals expressing a mammalian MOR and having a loss of function mutation in the bgg10 gene and methods of using the modified C. elegans animals to analyze the effectiveness of a compound in modulating opioid efficacy. In some embodiments, the modified C. elegans animals further expresses a heterologous hPCTHD1 gene or a heterologous PTR-25 gene. Methods of using the modified C. elegans animals comprise contacting the modified C. elegans animal with a compound or plurality of compounds and monitoring behavior of the animal in response to opioid, wherein decreased movement indicates increased effectiveness of the opioid.

A further understanding of the nature and advantages of the present invention may be realized by reference to the remaining portions of the specification and claims.

### BRIEF DESCRIPTION OF THE DRAWINGS

FIG. 1A-B. Forward genetic screen in C. elegans identifies PTR-25/Ptchd1 as a regulator of opioid tolerance. a, Schematic of C. elegans tgMOR opioid model. b, Experimental design for opioid tolerance assay using tgMOR C. elegans. Opioid dose-response analyzed before (1^{st}) and after (5^{th}) repeated exposures to vehicle or opioid.

FIG. 1C-E. Forward genetic screen in C. elegans identifies PTR-25/Ptchd1 as a regulator of opioid tolerance. c. Quantitation of fentanyl induced paralysis before (1^{st}) and after (5^{th}) repeated exposures. Decreased paralysis restored using higher fentanyl dose. Automated multi-worm tracker (MWT) used to acquire data. d. Summary of results from two-stage forward genetic screen for tgMOR tolerance mutants. Top chart represents first step where ˜900 tgMOR mutants were isolated with altered opioid sensitivity. Highlighted are 65 hypersensitive mutants (8% of all isolated mutants), false positives (black, 8% of mutants) and yposensitive mutants (85% of isolated mutants). Lower chart shows second-step where 27 tgMOR mutants with opioid hypersensitivity were screened for altered tolerance. Highlighted is 1 mutant, tgMOR; bgg10 that displayed impaired tolerance and 26 tgMOR hypersensitive mutants that did not show impaired tolerance. e. Quantitation of fentanyl (10 μM) induced paralysis with repeated drug exposure (left). Change in fentanyl response calculated as difference between 1^{st} and 5^{th} exposure (right). For panel c, statistical comparisons were performed by one-way ANOVA followed by Bonferroni's post hoc test. Data in panel e (right) was analyzed using a two-tailed unpaired Student's t-test and in panel e (left), by two-way ANOVA (right) followed by post hoc Bonferroni's test. In panels c and e error bars are SEM (n=12-24 wells, 48˜120 animals per genotype).

FIG. 1F-I. Forward genetic screen in C. elegans identifies PTR-25/Ptchd1 as a regulator of opioid tolerance. f. Dose-response curves showing that tgMOR animals develop tolerance upon repeated (5×) exposure to fentanyl (10 μM). Paralysis was measured and quantified as maximal possible effect (MPE) based on time to paralysis. g. Dose-response curves showing impaired tolerance in tgMOR; bgg10 animals after repeated (5×) exposure to fentanyl (10 μM). h, Quantitation of fentanyl responsiveness (EC₅₀ values for f and g) showing tgMOR; bgg10 animals do not develop tolerance and are hypersensitive. i, Topology of C. elegans PTR-25 and human PTCHD1. SSD (sterol sensing domain), EC (extracellular), IC (intracellular). For panel h, statistical comparisons were performed by one-way ANOVA followed by Bonferroni's post hoc test. Error bars are SD (n=3 replicates, 16˜20 animals per dose per replicate). ** p<0.01, *** p 0.001

FIG. 1J-M. Forward genetic screen in C. elegans identifies PTR-25/Ptchd1 as a regulator of opioid tolerance. j. Dose-response curves showing impaired tolerance in tgMOR; ptr-25 CRISPR animals after repeated (5×) exposure to fentanyl (10 μM). k. Dose-response curves showing transgenic expression of PTR-25 rescues impaired tolerance in tgMOR. bgg10 animals after repeated (5×) exposure to fentanyl (10 μM). l, Quantitation of fentanyl responsiveness (EC₅₀ values for j and k) showing PTR-25 impacts tolerance and sensitivity. m, Quantitation of fentanyl responsiveness (EC₅₀ values) showing human PTCHD1 rescues hypersensitivity of tgMOR; bgg10 mutants. For panel j, statistical comparisons were performed by one-way ANOVA followed by Bonferroni's post hoc test. Error bars are SD (n=3 replicates, 16˜20 animals per dose per replicate). ** p<0.01, *** p 0.001

FIG. 2A-B. Transgenics validates PTR-25/PTCHD1 as causal gene mutated in tgMOR; bgg10 animals and shows that human PTCHD1 functions in C. elegans. Time course of fentanyl application for indicated genotypes: a, TgMOR; ptr-25 CRISPR animals phenocopied hypersensitivity in tgMOR; bgg10 mutants; b, Native rescue with PTR-25 restored opioid sensitivity of tgMOR; bgg10 animals. Arrows indicate application of fentanyl (10 μM). Shown is SEM (n=12 wells, 48˜60 animals per genotype) analyzed by two-way ANOVA with Bonferroni's post hoc test. *** p<0.001, * p<0.05.

FIG. 2C-D. Transgenics validates PTR-25/PTCHD1 as causal gene mutated in tgMOR; bgg10 animals and shows that human PTCHD1 functions in C. elegans. Time course of fentanyl application for indicated genotypes; c, Pan-neuronal rescue (rab-3 promoter) with PTR-25 reversed opioid sensitivity of tgMOR. bgg10 mutants; d, transgenic expression of human PTCHD1 using the native ptr-25 promoter rescues opioid hypersensitivity of tgMOR; bgg10 animals. Arrows indicate application of fentanyl (c 10 μM; d 5 μM). Shown is SEM (n=12 wells, 48˜60 animals per genotype) analyzed by two-way ANOVA with Bonferroni's post hoc test. *** p<0.001, * p<0.05.

FIG. 2E. Transgenics validates PTR-25/PTCHD1 as causal gene mutated in tgMOR; bgg10 animals and shows that human PTCHD1 functions in C. elegans. e, Dose-response curves with fentanyl showing transgenic human PTCHD1 decreased opioid sensitivity of tgMOR; bgg10 animals. Shown is SEM (n=12 wells, 48˜60 animals per genotype) analyzed by two-way ANOVA with Bonferroni's post hoc test. *** p<0.001, * p<0.05

FIG. 3A-C. Knockout of Ptchd1 in mice enhances opioid efficacy and eliminates tolerance. a, Evaluation of morphine reward by conditioned place preference test. Mice received increasing doses of morphine. Data are from 4-11 male mice per genotype. b, Evaluation of analgesia by hot plate assay. Mice received increasing doses of morphine. Data are from 5-7 male mice per genotype. c, Evaluation of analgesia by tail immersion assay. Mice received increasing doses of morphine. Data are from 5-9 male mice per genotype. The first bar in each pair is WT and the second bar in each pair is Ptchd1 KO. Statistical comparisons were performed by 2-way ANOVA and Sidak's post hoc comparison. * p<0.05, ** p<0.01, *** p<0.001, **** p<0.0001. Mean values with S.E.M. errors are shown.

FIG. 3D-G. Knockout of Ptchd1 in mice enhances opioid efficacy and eliminates tolerance. d, Evaluation of analgesic tolerance by hot plate assay. Mice received repeated morphine injections (20 mg/kg) over 5 days. Data are from 5 male mice per genotype. e, Quantification of analgesic efficacy reduction as a difference in the MPE reduction between session 1 and 5 from panel d. f, Evaluation of opioid induced hyperalgesia by hot plate assay. Baseline response latencies of mice receiving repeated morphine injections (in panel d) prior to drug administration are plotted. g. Quantification of opioid induced hyperalgesia as a difference in the baseline latencies between session 1 and 5 from panel f. For panels d and f the statistical comparisons were performed by 2-way ANOVA and Bonferroni's post hoc comparison. For panels e and g, statistical comparisons were performed by unpaired two tailed Student's t-test. * p<0.05, ** p<0.01, *** p<0.001, **** p<0.0001. Mean values with S.E.M. errors are shown.

FIG. 3H-K. Knockout of Ptchd1 in mice enhances opioid efficacy and eliminates tolerance. h, Evaluation of analgesic tolerance by tail immersion assay. Mice received repeated morphine injections (20 mg/kg) over 5 days. Data are from 5-9 male mice per genotype. i, Quantification of analgesic efficacy reduction as a difference in the MPE reduction between session 1 and 5 from panel h. j, Evaluation of opioid induced hyperalgesia by tail immersion assay. Baseline response latencies of mice receiving repeated morphine injections (in panel h) prior to drug administration are plotted. k. Quantification of opioid induced hyperalgesia as a difference in the baseline latencies between session 1 and 5 from panel j. For panels h, and j the statistical comparisons were performed by 2-way ANOVA and Bonferroni's post hoc comparison. For panels i and k. statistical comparisons were performed by unpaired two tailed Student's t-test. * p<0.05, ** p<0.01, *** p<0.001, **** p<0.0001. Mean values with S.E.M. errors are shown.

FIG. 3L-M. Knockout of Ptchd1 in mice enhances opioid efficacy and eliminates tolerance. l. Dose-response profile of analgesic tolerance. Scheme reflects morphine administration protocol. Mice were evaluated by tail immersion assay before and after tolerance-inducing repeated injections of morphine (20 mg/kg). Data are from 6-7 male mice per genotype. m, Evaluation of somatic withdrawal signs and weight loss following precipitated withdrawal from chronic morphine administration. Data are from 11-12 male mice per genotype (First bar in each pair is WT; second bar in each pair is Ptchd1 KO). In panel l data was analyzed by one-way ANOVA with Tukey's post hoc comparison. In panel m, statistical comparisons were performed by unpaired two tailed Student's t-test. * p<0.05, ** p<0.01, *** p<0.001, **** p<0.0001. Mean values with S.E.M. errors are shown.

FIG. 4A. Ptchd1 regulates opioid efficacy and response desensitization. a, Macroscopic image illustrating distribution of MOR-mCherry (red), PTCHD1-YFP (green), and DAPI (blue) in a representative coronal section through the mouse midbrain.

FIG. 4B-C Ptchd1 regulates opioid efficacy and response desensitization. b, High magnification images examining MOR and Ptchd1 coexpression in the ventral tegmental area (VTA). c, Quantification of MOR and Ptchd1 coexpression in the VTA. For panel c, n=239 neurons from 13 mice. * p<0.05, ** p<0.01, *** p<0.001. **** p<0.0001. Mean values with S.E.M. errors are shown.

FIG. 4D. Circuit diagram of the VTA marking recording sites for FIGS. 4E-H.

FIG. 4E-H. Ptchd1 regulates opioid efficacy and response desensitization. e, Traces of averaged GIRK current evoked by the application of 10 μM DAMGO in GABA neurons (light outline is experimental error). Note that responses were normalized by capacitance for averaging. Amplitudes ranged from 19 pA to 333 pA across genotypes and conditions. f, Quantification of maximal GIRK mediated response amplitudes in GABA neurons across a range of DAMGO concentration. Data are from 6-11 neurons obtained from 3-6 mice (First bar in each pair is WT; second bar in each pair is Ptchd1 KO). g. Traces of averaged GIRK currents evoked by repeated application of DAMGO (1 μM) in Ptchd1 KO and control GABA neurons. The response to the second application is diminished due to MOR desensitization. h, Quantification of desensitization between paired first and second DAMGO-provoked GIRK currents. Data are from 6-11 neurons from 3-6 mice (First bar in each pair is WT; second bar in each pair is Ptchd1 KO). For panel f, the statistical comparisons were performed by unpaired two-tailed student's t-test. For panels e-h, n=6-11 neurons from 3-6 mice. * p<0.05, ** p<0.01, *** p<0.001, **** p<0.0001. Mean values with S.E.M. errors are shown.

FIG. 4I-J. Ptchd1 regulates opioid efficacy and response desensitization. i. Macroscopic image illustrating distribution of MOR-mCherry (red), PTCHD1-YFP (green), and Nissl (white) in a representative DRG section. j, Quantification of MOR and Ptchd1 coexpression in DRG sections. For panel j, n=1126 neurons from 3 mice. * p<0.05. ** p<0.01, *** p<0.001, **** p<0.0001. Mean values with S.E.M. errors are shown.

FIG. 4K-N. Ptchd1 regulates opioid efficacy and response desensitization. k, Diagram of a DRG nociceptor for FIGS. 4I-N. l. Quantification of rheobase at baseline and in response to repeated application of 10 μM morphine in Pichd1 KO and control DRG nociceptors. m. Quantification of morphine responses as change in rheobase from baseline to 1^{st} application of 10 μM morphine, and washout to 2^{nd} application of 10 μM morphine for Ptchd1 KO and control DRG nociceptors (First bar in each pair is WT; second bar in each pair is Ptchd1 KO). n, Quantification of desensitization between first and second morphine responses in Ptchd1 KO and control DRG nociceptors. For panel m, the results were analyzed by a mixed-model ANOVA with Holm-Šídak post-hoc test. For panels l and m, n=18-19 neurons from 4 mice. * p<0.05. ** p<0.01, *** p<0.001, **** p<0.0001. Mean values with S.E.M. errors are shown.

FIG. 5A-C. Molecular mechanism of Ptchd1 action in MOR regulation. a, Representative immunocytochemistry images of HEK293T/17 cells expressing HA-MOR (green), Na⁺/K⁺ ATPase (red, plasma membrane marker) and PTCHD1-FLAG (cyan). White lines indicate analysis area. b, Spatial analysis of MOR fluorescence intensity distribution across white line indicated in a. c, Quantification of MOR content on the plasma membrane (PM) or internal membranes (Internal) from 12 cells. For panel c statistical comparisons were performed by an unpaired two tailed Student's t-test. All data are presented as mean #S.E.M. ns, p>0.05, *p<0.05, ** p<0.01, *** p<0.001, **** p<0.0001.

FIG. 5D. Experimental design of assays determining cell surface abundance, internalization and recycling of MOR. Addition of LgBiT to HiBiT-MOR expressing cells forms a functional nanoluciferase (nLuc) enzyme. DAMGO treatment causes internalization and luminescence quenching. Naloxone (Nlx) blocks MOR signaling and results in recycling of internalized receptor.

FIG. 5E-H. Molecular mechanism of Ptchd1 action in MOR regulation. e, Quantitative analysis of Ptchd1 effect on MOR surface abundance in intact cells from 9 independent experiments. Varying levels (1×, 2×) of Ptchd1 cDNA were used for the transfection. f, Quantitative analysis of Ptchd1 effect on total MOR levels following cell lysis from 5 independent experiments. g, Quantification of Ptchd1 effect on DAMGO-induced MOR internalization. Loss of nLuc signal 17.5 min following addition of 10 μM DAMGO was determined in 9 independent experiments. h, Quantification of Ptchd1 effect on MOR recycling. Restoration of nLuc signal 60 min after treatment with 100 μM Nlx was determined in 6 experiments and normalized to surface abundance after internalization. The results were analyzed by two-way ANOVA with Dunett's post-hoc test. All data are presented as mean±S.E.M. ns, p>0.05, *p<0.05, ** p<0.01, *** p<0.001, **** p<0.0001.

FIG. 5I. Experimental design of β-arrestin recruitment assay. Addition of DAMGO induces recruitment of LgBiT-tagged β-arr2 to MOR-SmBIT to form a functional nLuc enzyme.

FIG. 5J-L. Molecular mechanism of Ptchd1 action in MOR regulation. j, Time course of Ptchd1 effect on recruitment of β-arr2 to MOR determined in 7 independent experiments. k, Quantification of the effect of Ptchd1 on the maximum fold-change of β-arr2 recruitment from data in j. l, Quantification of the initial rate of β-arr2 recruitment to MOR from data in j. Data are from 5 independent experiments. In panels k and l the results were analyzed by two-way ANOVA with Dunett's post-hoc test. All data are presented as mean±S.E.M. ns, p>0.05, *p<0.05, ** p<0.01, *** p<0.001, **** p<0.0001.

FIG. 6A. Schematic illustration of assay experimental design for determining cholesterol content in the plasma membrane. The GFP-D4-YDA cholesterol sensor binds plasma membrane cholesterol in close proximity to membrane-bound GRK3-Nluc-KRas forming a BRET pair. Quantification indicates the effects of Pitchd1 KO and other manipulations in the cholesterol BRET assay. Significance was determined using a one-way ANOVA with Tukey's post-hoc test. * p<0.05, ** p<0.01. *** p<0.001, **** p<0.0001. Mean values with S.E.M. errors are shown.

FIG. 6B-E. Role of cholesterol in mediating the effects of Pichd1 on MOR desensitization. b, Quantification of the effects of Ptchd1 KO on DAMGO-mediated MOR internalization following cholesterol depletion by 4 mM MBCD. c. Quantification of the effects of Ptchd1 KO on DAMGO-mediated MOR internalization following treatment with 200 μg/ml cholesterol. d, Quantification of rheobase from baseline through two treatments of 10 μM morphine and recovery, in control (Ptchd1 heterozygous) DRG with or without treatment with 200 μg/mL cholesterol. e, Quantification of morphine responses as change in rheobase from baseline to 1^{st} application of 10 μM morphine, and washout to 2^{nd} application of 10 μM morphine in control DRG with or without treatment with 200 μg/mL cholesterol. In panel b significance was determined using a one-way ANOVA with Tukey's post-hoc test. For panel e, significance was determined using a two-way ANOVA with Holm-Šídák post-hoc test. For panels d and e, n=8-15 neurons from 3 mice. * p<0.05, ** p<0.01, *** p<0.001, **** p<0.0001. Mean values with S.E.M. errors are shown.

FIG. 6F-I. Role of cholesterol in mediating the effects of Ptchd1 on MOR desensitization. f, Quantification of desensitization between first and second morphine responses in e. g. Quantification of rheobase from baseline through two treatments of 10 μM morphine and recovery, in Pichd1 KO DRG with or without 4 mM MBCD treatment. h, Quantification of morphine responses as change in rheobase from baseline to 1^{st} application of 10 μM morphine, and washout to 2^{nd} application of 10 μM morphine in Ptchd1 KO DRG with or without 4 mM MBCD treatment. i, Quantification of desensitization between first and second morphine responses in h. For panel h, significance was determined using a two-way ANOVA with Holm-Šídák post-hoc test. For panels f and i. significance was determined with an unpaired two-tailed Student's t-test. For panels f, n=8-15 neurons from 3 mice. For panels g, h, and i, n=18-15 neurons from 3-4 mice. * p<0.05, ** p<0.01, *** p<0.001, **** p<0.0001. Mean values with S.E.M. errors are shown.

FIG. 7. Proposed mechanism for Ptchd1 regulation of MOR signaling and opioid tolerance. Ptchd1 acts to deplete membrane cholesterol which in turn affects MOR dynamics impeding its trafficking to the surface and internalization following activation by opioids. When trapped on the surface due to Ptchd1 action, MOR fails to undergo resensitizing internalization/recycling leading to prolonged signaling which triggers desensitization and tolerance. Red inhibitory bars and red arrows indicate negative regulatory functions and effects on reducing membrane cholesterol levels, respectively.

FIG. 8A-C. Behavioral characterization of opioid responsiveness of Pichd1 KO mice. a, Analysis of time course of morphine analgesia by hot plate assay upon systemic morphine administration. Mice received 10 mg/kg morphine. Data are from 6-7 male mice per genotype. b, Evaluation of morphine induced analgesia in hot plate assay upon intracerebroventricular (ICV) injection of morphine. Mice received 3.5 nmol of morphine. Data are from 7 male mice per genotype. c. Analysis of time course of morphine analgesia in the hot plate assay following ICV injection of morphine. For panels a and c statistical comparisons were performed by 2-way ANOVA and Bonferroni's post hoc comparison. For panel b. statistical comparisons were performed by multiple unpaired two tailed Student's t-test with Bonferroni's post hoc. * p<0.05, ** p<0.01, *** p<0.001. **** p<0.0001. Mean values with S.E.M. errors are shown.

FIG. 8D-G. Behavioral characterization of opioid responsiveness of Pichd1 KO mice. d. Evaluation of analgesia by hot plate assay with systemic fentanyl administration. Mice received 0.1 mg/kg of fentanyl. Data are from 7 male mice per genotype. e, Analysis of time course of fentanyl analgesia by hot plate assay from the experiment in panel d. f, Evaluation of chronic analgesic tolerance by hot plate assay. Mice received repeated fentanyl injections (0.15 mg/kg) over 10 days. Data are from 7 male mice per genotype. g. Quantification of analgesic efficacy reduction as a difference in the MPE reduction between session 1 and 10 from panel f. For panels e and f, statistical comparisons were performed by 2-way ANOVA and Bonferroni's post hoc comparison. For panel d, statistical comparisons were performed by multiple unpaired two tailed Student's t-test with Bonferroni's post hoc. For panel g, statistical comparisons were performed by unpaired two tailed Student's t-test. * p<0.05, ** p<0.01, *** p<0.001, **** p<0.0001. Mean values with S.E.M. errors are shown.

FIG. 8H-K. Behavioral characterization of opioid responsiveness of Pichd1 KO mice. h, Evaluation of opioid induced hyperalgesia by hot plate assay. Baseline response latencies of mice receiving repeated fentanyl injections prior to drug administration are plotted. i, Quantification of opioid induced hyperalgesia as a difference in the baseline latencies between session 1 and 10 from panel h. j. Evaluation of acute analgesic tolerance by hot plate assay. Mice received repeated morphine injections (20 mg/kg) 120 minutes apart. Data are from 5-7 male mice per genotype. k. Quantification of analgesic efficacy reduction as a difference in the MPE reduction between session 1 and 2. For panel h, statistical comparisons were performed by 2-way ANOVA and Bonferroni's post hoc comparison. For panel j, statistical comparisons were performed by multiple unpaired two tailed Student's t-test with Bonferroni's post hoc. For panels i and k, statistical comparisons were performed by unpaired two tailed Student's t-test. * p<0.05, ** p<0.01. *** p<0.001, **** p<0.0001. Mean values with S.E.M. errors are shown.

FIG. 9A. Electrophysiological characterization of opioid responses in Ptchd1 KO neurons. a. Quantification of both baseline sIPSC frequencies and their inhibition in DA neurons upon sequential applications of increasing concentrations of DAMGO. Data are from 6-7 neurons from 3 mice (First bar in each pair is vehicle; second bar in each pair is Ptchd1 KO). N=6-7 neurons from 3 mice. Statistical comparison was performed by 2-way ANOVA with Holm-Šídák post-hoc test. * p<0.05, ** p<0.01, *** p<0.001, **** p<0.0001. Mean values with S.E.M. errors are shown.

FIG. 9B-D. Electrophysiological characterization of opioid responses in Ptchd1 KO neurons. b, Quantification of rheobase from baseline. through two applications of 10 mM morphine and recovery. c. Quantification of 10 mM morphine responses as the difference in rheobase (First bar in each pair is vehicle; second bar in each pair is Ptchd1 KO). d, Quantification of desensitization between first and second morphine responses. For panel d, the statistical comparison was performed by an unpaired two-tailed student's t-test. N=8-9 neurons from 4 mice. * p<0.05. ** p<0.01, *** p<0.001, **** p<0.0001. Mean values with S.E.M. errors are shown.

FIG. 10A-B. Cell biological characterization of Ptchd1 actions. a, Modulation of DAMGO mediated-βarr2-LgBiT recruitment to MOR-SmBIT following incubation with 4 mM MBCD or 200 μg/mL cholesterol. Data are mean #S.E.M. of 6-7 experiments. b, Quantification of the maximum fold change and p, the initial recruitment rate from panel n. Significance was determined using a one-way ANOVA and Dunnett's post-hoc. ns, p>0.05, *p<0.05, ** p<0.01, *** p<0.001. **** p<0.0001.

FIG. 11A-B. Effect of simvastatin treatment on morphine tolerance in hot plate test and somatic withdrawal. A. C57/B16 mice (males and females; 10-12 per group) were subjected to oral gavage with indicated dose of simvastatin or vehicle (water) once daily for 6 consecutive days. On day 7 analgesic tolerance was tested by injecting mice with morphine for 5 days. In each of these days, simvastatin treatment continued at the same dose. B, C57/B16 mice were injected with escalating doses of morphine as described followed by naloxone precipitation of withdrawal and scoring the withdrawal signs as indicated. *** p<0.01 and **** p<0.001 Two-way ANOVA with Bonferroni's post hoc test for differences between treatments (First bar in each pair is vehicle. Second bar in each pair is simvastatin (10 mg/kg)).

### DETAILED DESCRIPTION

### A. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease. i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with a disease, disorder, or condition or those in which the disease, disorder, or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

“Opioid” includes opioid drugs and opioid-related drugs or compounds that are members of a class of drugs either derived from, or chemically similar to, compounds found in opium poppies. Examples of opioids include legal prescription painkillers like oxycodone (OXYCONTIN®), hydrocodone (VICODIN®), morphine, codeine, dihydrocodeine, fentanyl, and the like, and illegal drugs such as opium and heroin. Opioids also include antagonist drugs such as naloxone, and endogenous peptides such as endorphins. In some embodiments, opioid compounds can also include partial agonists of MOR, e.g., buprenorphine and methadone.

An “opioid use disorder” is a substance use disorder (persistent use of a drug despite harm and adverse consequences) relating to the use of an opioid. Signs of the disorder include a strong desire to use opioids, impaired control over its use, increased tolerance to opioids, persistent use despite harmful consequences, trouble reducing use, and withdrawal symptoms with discontinuation. Opioid withdrawal symptoms include, but are not limited to, nausea, muscle aches, diarrhea, trouble sleeping, agitation, and a low mood. Addiction and dependence are components of a substance use disorder.

Withdrawal, withdrawal symptoms, or withdrawal syndrome refers to a collection of symptoms and the degree of severity which the symptoms occur on cessation or abrupt reduction of use of a psychoactive substance (e.g., an opioid drug) that has been taken repeatedly, usually for a prolonged period and/or in high doses. The syndrome may be accompanied by signs of physiological and/or emotional disturbance. A withdrawal symptom is one of the indicators of a dependence syndrome.

The μ-opioid receptors are a class of opioid receptors with a high affinity for endogenous opioid peptides enkephalins and beta-endorphin, but a low affinity for dynorphins. MOR is encoded by the OPRM gene. They are also referred to as μ-opioid peptide (MOR) receptors. The prototypical exogenous MOR agonist is morphine, the primary psychoactive alkaloid in opium. It is an inhibitory G-protein coupled receptor that activates several inhibitory G protein subunits, including Giα, Goα, Gzα, and Gβγ (G beta-gamma), inhibiting activity of adenylate cyclase to lower cAMP levels and several ion channels to reduce neuronal excitability and synaptic transmission. Activation of the MOR by an agonist, such as morphine, causes analgesia, sedation, slightly reduced blood pressure, itching, nausea, euphoria, decreased respiration, miosis (constricted pupils), and decreased bowel motility often leading to constipation. Some of these effects, such as analgesia, sedation, euphoria, itching, and decreased respiration, tend to lessen with continued use as tolerance develops. As with other G protein-coupled receptors, signaling by the MOR is terminated through several different mechanisms, which are upregulated with chronic use, leading to rapid tachyphylaxis.

Ptchd1 (Patched Domain Containing Protein 1) is a protein encoded by the Ptchd1 gene. The Ptchd1 protein is a membrane protein with a patched domain and a sterol sensing domain. It is similar to Drosophila proteins which act as receptors for the morphogen sonic hedgehog. Ptchd1 activity leads to decreased cholesterol in neural tissues. Diseases associated with Ptchd1 include Autism X-Linked 4 and Non-Syndromic X-Linked Intellectual Disability.

A Ptchd1 antagonist is a compound that down-regulates or inhibits Ptchd1 gene expression, Ptchd1 mRNA levels, or cellular activity of Ptchd1, or down-regulates or inhibits Ptchd2 pathway activity.

A Ptchd1 pathway antagonist is a compound that counteracts, inhibits, or antagonizes Ptchd1 or a downstream affect of Ptchd 1 activity. Ptchd1 pathway antagonists include compounds that increase cholesterol in neuronal tissues.

“Orthologs” are genes and products thereof in different species that evolved from a common ancestral gene by speciation and retain the same or similar function. An ortholog is a gene that is related by vertical descent and is responsible for substantially the same or identical functions in different organisms. For example, mouse GPR139 and human GPR139 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

An “analog” refers to a molecule that structurally resembles a reference molecule (e.g., a GPR139 antagonist) but which has been modified in a targeted and controlled manner, by replacing a specific substituent of the reference molecule with an alternate substituent. Compared to the reference molecule, an analog would be expected, by one skilled in the art, to exhibit the same or similar utility. Synthesis and screening of analogs to identify variants of known compounds having improved characteristics (such as higher binding affinity for a target molecule) is an approach that is well known in pharmaceutical chemistry.

Sequence identity can be determined by aligning sequences using algorithms, such as BESTFIT, FASTA, and TFASTA in the Wisconsin Genetics Software Package Release 7.0. Genetics Computer Group. 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick or other non-traditional types. A percent complementarity indicates the percentage of bases, in a contiguous strand, in a first nucleic acid sequence which can form hydrogen bonds (e.g., Watson-Crick base pairing) with a second nucleic acid sequence (e, g., 5, 6, 7, 8, 9, 10 out of 10 being 50%, 60%, 70%, 80%, 90%, and 100% complementary). Percent complementarity is calculated in a similar manner to percent identify.

A “derivative” of a first compound is a compound that has a three dimensional structure that is similar to at least a part of the first compound. In some embodiments, a derivative is a compound that is derived from, or imagined to derive from, another compound such as by substitution of one atom or group with another atom or group. In some embodiments, derivatives are compounds that at least theoretically can be formed from a common precursor compound.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

### B. Increasing Opioid Therapy Efficacy

Opioid analgesics offer pain management but have substantial abuse liability. Opioids produce clinically significant effects via the MOR, a member of the G protein Coupled Receptor (GPCR) family. Saturation of MOR as a drug target presents a pressing need to discover new modifiers that alter MOR signaling outcomes.

We show that Ptchd1 plays a role in MOR activity and animal responses to opioid therapy. Loss of Ptchd1 in model animals reduces opioid-induced desensitization of neurons across the nervous system and makes them more susceptible to opioid inhibition. Mice lacking Ptchd1 display augmented responses to opioids and fail to develop tolerance. We also show that the Ptchd1 pathway affects MOR trafficking via effects on cholesterol.

Described are methods of modulating opioid efficacy or safety by targeting the Ptchd1 gene, the Ptchd1 protein, or the Ptchd1 pathway. The Ptched1 and/or the Ptchd1 pathway antagonists, and pharmaceutical compositions containing the Ptchd1 and/or Ptchd1 pathway antagonists are described.

In some embodiments, methods for modulating analgesic response in subjects who are taking opioid related drugs are provided. Administering a Ptched1 antagonist and/or a Pichd1 pathway antagonist to a subject can be used to increase efficacy of an opioid drug and/or diminishing the dependence-causing liability of the opioid drug. Such use can be used to treat, prevent, or reduced the likelihood of addiction. The subject can be, but is not limited to, a subject that is an acute user or a chronic user of opioids. In some embodiments, a Ptched1 and/or Ptchd1 pathway antagonist enhances endogenous opioid signaling. Enhancing endogenous opioid signaling can be used to provide or enhance analgesic response, such as by an opioid. Increased efficacy or safety of an opioid or opioid therapy includes, but is not limited to, enhancing maximal response to the opioid, increasing response to a given dose of opioid, decreasing the amount of the opioid required to provide an effective analgesic response, increasing duration of analgesic response to the opioid, decreasing tolerance of the opioid, increasing safety of an opioid therapeutic, decreasing risk of addiction or dependence on the opioid, and suppressing or decreasing withdrawal symptoms. Decreasing the amount of the opioid required to provide an effective analgesic response can be at an initial dose or one or more subsequent doses.

In some embodiments, methods are provided for modulating cellular activities mediated by the MOR signaling, comprising administering to a subject a Ptched1 antagonist and/or a Ptchd1 pathway antagonist. Antagonism of Ptched1 or Ptchd1 pathway activity, using a described Ptched1 antagonist or a Ptchd1 pathway antagonist, enhances MOR signaling and sensitivity to opioid compounds.

In some embodiments, a subject in need of treatment, such as opioid treatment, is administered a described Ptched1 or a Ptchd1 pathway antagonist in addition to the opioid, to promote MOR mediated signaling activity and enhance opioid efficacy. The Ptched1 or Ptchd1 pathway antagonist can be administered prior to administration of the opioid, concurrent with opioid administration, or subsequent to opioid administration. The Ptched1 or Ptchd1 pathway antagonist may be administered to the subject to improve pain relief associated with opioid treatment. The subject can be, but is not limited to, a human.

In some embodiments, a described Ptched1 or Ptchd1 pathway antagonist is administered to a subject affected by chronic use of an opioid to diminish dependence and/or ameliorate withdrawal symptoms. The Ptched1 or Ptchd1 pathway antagonist can be administered prior to administration of the opioid, concurrent with opioid administration, subsequent to opioid administration (after the subject has stopped taking the opioid), or subsequent to a reduction in opioid use by the subject. The subject can be, but is not limited to, a human.

In some embodiments, a described Ptched1 or Ptchd1 pathway antagonist is administered to a subject to reduce dependence on an opioid, control relapse, or suppress or ameliorate withdrawal symptoms, wherein the subject has been chronically taking one or more opioids or suffers from an opioid use disorder. The Ptched1 or Ptchd1 pathway antagonist can be administered to the subject prior to the subject discontinuing or reducing opioid drug use, concurrent with discontinuing or reducing opioid drug use, or after discontinuing or reducing opioid drug use. In some embodiments, the Ptched1 or Ptchd1 pathway antagonist is administered to a subject that has discontinued or reduced opioid use and is suffering from one or more withdrawal symptoms. The Ptched1 or Ptchd1 pathway antagonist can be administered to the subject for as long as the one or more withdrawal symptoms persist. The subject can be, but is not limited to, a human.

Subjects taking any opioid related drugs are amenable to treatment with the described Ptched1 or Ptchd1 pathway antagonists. The opioid drugs include, but are not limited to, morphine, synthetic opioid related compounds, methadone, oxycodone, hydrocodone, codeine, dihydrocodeine, pethidine, hydromorphone, heroin, opium, and fentanyl.

In some embodiments, a described Ptched1 or Ptchd1 pathway antagonist or a pharmaceutical composition containing the Ptched1 or Ptchd1 pathway antagonist is administered with an opioid drug. In some embodiments, a pharmaceutical composition containing a Ptched1 or Ptchd1 pathway antagonist further comprises an opioid drug.

In some embodiments, the Ptchd1 antagonist comprises a Ptchd1 nucleic acid-based inhibitor, wherein the nucleic acid-based inhibitor targets the Ptchd1 mRNA.

A nucleic acid-based inhibitor comprises any polynucleotide that is not translated into protein but whose presence in the cell alters decreases expression of a target gene. A nucleic acid-based inhibitor comprises a poly nucleotide, including poly nucleotides containing nucleotide analogs, containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. Nucleic acid-based inhibitor are selected from the group comprising: RNAi polynucleotides, siRNA, microRNA, dsRNA, RNA Polymerase II transcribed DNAs encoding siRNA or antisense oligonucleotides, RNA Polymerase III transcribed DNAs encoding siRNA or antisense oligonucleotides, ribozymes, antisense oligonucleotides, and antisense nucleic acid, which may be RNA, DNA, or artificial nucleic acid.

An RNA interference (RNAi) polynucleotide is a molecule capable of inducing RNA interference through interaction with the RNA interference pathway machinery of mammalian cells to degrade or inhibit translation of messenger RNA (mRNA) transcripts of a transgene a sequence specific manner. RNAi polynucleotides may be selected from the group comprising: siRNA, microRNA, double-strand RNA (dsRNA), short hairpin RNA (shRNA), and expression cassettes encoding RNA capable of inducing RNA interference, siRNA comprises a double stranded structure typically containing 15-50 base pairs and preferably 21-25 base pairs and having a nucleotide sequence identical (perfectly complementary) or nearly identical (partially complementary) to a sequence present in an expressed target gene or RNA within the cell. An siRNA may have overhangs, such as dinucleotide 3′ overhangs. An siRNA may be composed of two annealed polynucleotides or a single polynucleotide that forms a hairpin structure. An siRNA molecule comprises a sense region and an antisense region. In one embodiment, the siRNA of the conjugate is assembled from two oligonucleotide fragments wherein one fragment comprises the nucleotide sequence of the antisense strand of the siRNA molecule and a second fragment comprises nucleotide sequence of the sense region of the siRNA molecule. In another embodiment, the sense strand is connected to the antisense strand via a linker molecule, such as a polynucleotide linker or a non-nucleotide linker.

MicroRNAs (miRNAs) are small noncoding RNA gene products about 22 nucleotides long that direct destruction or translational repression of their mRNA targets. For miRNAs, the complex binds to target sites usually located in the 3′ UTR of mRNAs that typically share only partial homology with the miRNA. A “seed region”—a stretch of about seven (7) consecutive nucleotides on the S′ end of the miRNA that forms perfect base pairing with its target-plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region (Pillai et al, 2007).

An antisense oligonucleotide (ASOs) comprises an oligonucleotide having a nucleobase sequence that is complementary to a target nucleic acid or region or segment thereof. Antisense oligonucleotides include, but are not limited to: morpholinos, 2′-O-methyl polynucleotides, DNA, RNA and the like. In some embodiments, an antisense oligonucleotide is specifically hybridizable to a target nucleic acid or region or segment thereof. ASOs can comprise nucleobase sequence and optionally one or more additional features, such as a conjugate group or terminal group. ASOs may be single-stranded and double-stranded compounds. ASOs include, but are not limited to, oligonucleotides, ribozymes, gapmers, and morpholinos (peptide-conjugated phosphorodiamidate oligonucleotides (PPMOs) or simply phosphorodiamidate oligonucleotides (PMOs)). Antisense nucleic acids act by hybridization of an antisense nucleotide sequence to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target. Antisense nucleic acid can inhibit gene expression by reducing the levels of target RNA in a cell or by inhibiting translation, splicing, or activity of an RNA in a cell.

Gapmers are short DNA antisense oligonucleotide structures with RNA-like segments on both sides of the sequence. Gapmers are designed to hybridize to sequence in a target RNA and inhibit expression of the gene through the induction of RNase H cleavage. Binding of the gapmer to the target has a higher affinity due to the modified RNA flanking regions. The RNA flanking regions can have modified RNA nucleotides, such as, but not limited to, 2′-MOE (2′-O-(2-Methoxyethyl) modified nucleotides, or LNA (locked nucleic acid) modified nucleotides.

A nucleic acid-based inhibitor may be polymerized in vitro, recombinant, contain chimeric sequences, or derivatives of these groups. The nucleic acid-based inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

A nucleic acid-based inhibitor may be expressed from a DNA vector in a cell. Nucleic acid-based inhibitor expression cassettes can be transcribed in the cell to produce hairpin RNAs (including shRNA or miRNA), separate sense and anti-sense strand linear siRNAs, an antisense nucleic acid, or a ribozyme. Coding sequence for the nucleic acid-based inhibitor can be operatively linked to a promoter for expression of the nucleic acid-based inhibitor in a cell. The promoter can be a RNA polymerase III promoter of a RNA polymerase II promoter. A RNA polymerase III promoter can be, but is not limited to, a U6 promoter, a H1 promoter, or a tRNA promoter. A RNA polymerase II promoter can be, but is not limited to, a U1 promoter, a U2 promoter, a U4 promoter, a U5 promoter, an snRNA promoter, a microRNA promoters, or a mRNA promoter.

In some embodiments, a Pichd1 nucleic acid-based inhibitor comprises a double stranded ribonucleic acid (dsRNA) agent, such as an siRNA, for inhibiting expression of Ptchd1 in a cell, wherein the dsRNA agent comprises a sense strand and an antisense strand, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from a complement of SEQ ID NO: 1.

In some embodiments, a Pichd1 nucleic acid-based inhibitor comprises a double stranded ribonucleic acid (dsRNA) agent, such as an siRNA, for inhibiting expression of Ptchd1 in a cell, wherein the dsRNA agent comprises a sense strand and an antisense strand, wherein the sense strand comprises at least 15 contiguous nucleotides from the nucleotide sequence of SEQ ID NO: 1 and the antisense strand comprises at least 15 contiguous nucleotides from a complement of SEQ ID NO: 1.

In some embodiments, a Ptchd1 nucleic acid-based inhibitor comprises a double stranded ribonucleic acid (dsRNA) agent, such as an siRNA, for inhibiting expression of Pichd1 in a cell, wherein the dsRNA agent comprises a sense strand and an antisense strand, wherein the sense strand comprises at least 17, at least 19, at least 19, or at least 20 contiguous nucleotides from the nucleotide sequence of SEQ ID NO: 1 and the antisense strand comprises least 17, at least 18, at least 19, or at least 20 contiguous nucleotides from a complement of SEQ ID NO: 1.

ASOs can be designed using methods available in the art. In some embodiments, a Ptchd1 nucleic acid-based inhibitor comprises an ASO for inhibiting expression of Ptchd1 in a cell, wherein the ASO comprises at least 10 contiguous nucleotides differing by no more than 3 nucleotides from a complement of the nucleotide sequence of SEQ ID NO: 1. In some embodiments, the ASO comprises a gapmer. In some embodiments, a Ptchd1 nucleic acid-based inhibitor comprises an ASO for inhibiting expression of Ptchd1 in a cell, wherein the ASO comprises at least 10 contiguous nucleotides from a complement of the nucleotide sequence of SEQ ID NO: 1. In some embodiments, the ASO comprises a gapmer.

In some embodiments, a Ptchd1 nucleic acid-based inhibitor comprises an ASO for inhibiting expression of Ptchd1 in a cell, wherein the ASO comprises 10-20 contiguous nucleotides differing by no more than 3 nucleotides from a complement of the nucleotide sequence of SEQ ID NO:1. In some embodiments, the ASO comprises a gapmer.

In some embodiments, a Ptchd1 nucleic acid-based inhibitor comprises an ASO for inhibiting expression of Ptchd1 in a cell, wherein the ASO comprises 10-20 contiguous nucleotides from a complement of the nucleotide sequence of SEQ ID NO:1. In some embodiments, the ASO comprises a gapmer.

In some embodiments, a Ptchd1 nucleic acid-based inhibitor comprises an ASO for inhibiting expression of Ptchd1 in a cell, wherein the ASO comprises at least 10, 11, 12, 13, 14, 15, 16, 17, 18, 19, or 20 contiguous nucleotides from a complement of the nucleotide sequence of SEQ ID NO: 1. In some embodiments, the ASO comprises a gapmer.

In some embodiments, the Ptchd1 antagonist comprises a Ptchd1 CRISPR-based system. Ptchd1 CRISPR systems can be used to modify, disrupt, or mutate the Ptchd1 gene in a cell. A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. Other RNA-guided DNA endonuclease enzymes can be used, including derivatives of Cas9 used for gene editing. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the Pichd1 gene genomic region. The Cas protein can be introduced into the cell in the form of a protein or a nucleic acid (DNA or RNA) encoding the Cas protein (e.g., operably linked to a promoter expressible in the cell). The guide RNA can be introduced into the cell in the form of RNA or a DNA encoding the guide RNA (e.g., operably linked to a promoter expressible in the cell). In some embodiments, the Ptchd1 CRISPR system can be delivered to a cell via a viral vector.

A Ptchd1 CRISPR system is designed to target the Ptchd1 gene. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

A suitable guide sequence includes a 17-20 nucleotide sequences complementary to a target sequence in the Ptchd1 gene (SEQ ID NO: 1) that is unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in the Ptchd1 gene (SEQ ID NO: 1) or a complement thereof can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 1 or 2 mismatches when hybridized to the target sequence. In some embodiments, a mismatch, if present, is located distal to the PAM, in the 5′ end of the guide sequence.

PTCHD1 coding sequence-
SEQ ID NO: 1
atgctgcggcaggttctgcacaggggcttgaggacgtgtttctcccggctcggccacttcattgccagtcaccct
gtcttcttcgcctcggcgccggtgctcatctccatcctgctcggcgccagcttcagccgctaccaggtcgaggag
agcgtggagcacctgctggcgccccagcacagcctggccaagatcgagcgcaacctcgttaacagcctcttcccg
gtcaaccgctccaagcaccgtctctactcggacctgcagacccccgggcgctacggccgggtcatcgtcacctcc
ttccagaaagccaacatgctggaccagcatcacaccgacctgatcttaaagttgcatgctgctgtcaccaagatc
caggttccaaggcctggttttaattacacgtttgcccatatatgtatcctgaataatgataagacttgcatcgtg
gatgacatagtgcacgtcctggaagagctaaagaatgctcgggccaccaatcggaccaattttgctatcacatac
ccaaccactcacttaaaggacgggagggctgtgtacaatgggcaccagcttgggggcgtcactgtgcacagcaaa
gaccgggtgaaatctgcagaggccatccagctcacctactacctgcagtcaatcaacagtctcaatgacatggtg
gctgagaggtgggagtccagcttctgcgacactgtcagactgtttcagaaatccaacagcaaagtcaaaatgtac
ccttacacgtcctcctcactgagggaagatttccagaagaccagccgcgtatcagaacgttacctggtcaccagc
ctgattctggtggttaccatggccatcctgtgttgctctatgcaggactgcgtccgcagcaaaccctggctaggc
ctgctcggattggtgaccataagcctggccactctcactgcagccgggatcatcaatcttactggtgggaaatat
aattccaccttcctgggagtccctttcgtcatgctaggtcatggattatatgggacttttgaaatgttatcctcc
tggaggaaaactagagaagaccaacatgttaaagagagaactgcagcagtctatgcagactccatgctctccttt
tctc=caccactgccatgtacctggtcacctttggcataggggccagccctttcacgaacattgaggcagccagg
atttcctgctgcaattcctgtattgcaatcttcttcaactacctctatgtactctcgttttatggttccagccta
gtgt=cactggctacatagaaaacaattaccagcatagtatcttctgtagaaaagtcccaaagcctgaggcattg
caggagaagccggcatggtacaggtttctcctgacggccagattcagtgaggacacagctgaaggcgaggaagcg
aacacttacgagagtcacctattggtatgtttcctcaaacgctattactgtgactggataaccaacacctatgtc
aagccttttgtagttctcttttaccttatttatatttcctttgccttaatgggctatctgcaggtcagtgaaggg
tcagaccttagtaacattgtagcaaccgcgacacaaaccattgagtacactactgcccagcaaaagtacttcagc
aactacagtcctgtgattgggttttacatatatgagtctatagaatactggaacactagtgtccaagaagatgtt
ctagaatacaccaaggggtttgtgcggatatcctggtttgagagctatttaaattaccttcggaaactcaatgta
tccactggcttgcctaagaaaaatttcacagacatgttgaggaattcctttctgaaagcccctcaattttcacat
tttcaagaggacatcatcttctctaaaaaatacaatgatgaggtcgatgtagtggcctccagaatgtttttggtg
gccaagaccatggaaacaaacagagaagaactctatgatctcttggaaaccctgaggagactttctgtcacctcc
aaggtgaagttcatcgtcttcaatccgtcctttgtatacatggatcgatatgcctcctctctgggagcccccctg
cacaactcctgcatcagtgctttgttcctgctcttcttctcggcattcctggtggcagattcactgattaacgtc
tggatcactctcacagttgtgtccgtggagtttggagtgataggtttcatgacattatggaaagtagaactggac
tgcatttctgtgctatgcttaatttatggaattaastacacaattgacaattgtgctccaatgttatccacattt
gttctgggcaaggatttcacaagaactaaatgggtaaaaaatgccctggaagtgcatggggtagctattttacag
agttacctctgctatattgttggtctgattcctcttgcagctgtgccttcaaatctgacctgtacactgttcagg
tgcttgtttttaatagcatttgtcaccttctttcactgctttgccattttacctgtgatactgactttcctgcca
ccctctaagaaaaaaaggaaagagaagaaaaatcc_gagaaccgggaggaaattgagtgtgtagaaatggtagat
atcgatagtacccgtgtggttgaccaaattacaacagtg

In some embodiments, the Ptchd1 pathway antagonist comprises a compound that increases cholesterol in neuronal tissue. A Ptchd1 pathway antagonist can be, but is not limited to, a statin, a cholesterol absorption inhibitor, a PCSK9 inhibitor, a citrate lyase inhibitor, a bile acid sequestrant, a HDL-C raising therapeutic, a fibrate, niacin, an omega-3 fatty acid, an APOE-based therapeutic, or a ABCA1 cholesterol transporter-based therapeutic.

A Cholesterol absorption inhibitor can be, but is not limited to, ezetimibe. A PCSK9 inhibitor can be, but is not limited to, alirocumab, or evolocumab. A citrate lyase inhibitor can be, but is not limited to, bempedoic acid or bempedoic acid-ezetimibe. A bile acid sequestrant can be, but is not limited to, cholestyramine, colesevelam, and colestipol. An HDL-C raising therapeutic can be, but is not limited to, rosuvastatin, simvastatin, and atorvastatin. A fibrate can be, but is not limited to, fenofibrate and gemfibrozil. An omega-3 fatty acid can be, but is not limited to, lovaza, omacor, or vascepa. An APOE-based therapeutic can be, but is not limited to, CN-105, CS-6253, a phthalazinone, a pyrazoline, an anti-APOE antibody, an APOE4 ASO (antisense oligonucleotide), an APOE4 RNAi polynucleotide, bexarotene, probucol, AGB101, or epigallocatechin gallate. An ABCA1 cholesterol transporter-based therapeutic can be, but is not limited to, probucol.

The compounds and pharmaceutical compositions disclosed herein can be administered to a subject once per day, more than once a day, for example, 2, 3, 4, 5, or 6 times a day, or as needed.

In some embodiments, the Ptched1 or Ptchd1 pathway antagonists are formulated with one or more pharmaceutically acceptable excipients (including vehicles, carriers, diluents, and/or delivery polymers), thereby forming a pharmaceutical composition or medicament suitable for in vivo delivery to a subject, such as a human.

A pharmaceutical composition or medicament includes a pharmacologically effective amount of the active compound and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support, or enhance stability, bioavailability or patient acceptability of the API, c) assist in product identification, and/or d) enhance any other attribute of the overall safety and effectiveness of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants, such as preservatives, wetting agents, emulsifying agents, and dispersing agents. A carrier may also contain isotonic agents, such as sugars, poly alcohols, sodium chloride, and the like into the compositions.

The pharmaceutical compositions can contain other additional components commonly found in pharmaceutical compositions. Such additional components can include, but are not limited to: anti-purities, astringents, local anesthetics, or anti-inflammatory agents (e.g., antihistamine, diphenhydramine, etc.).

The phrase “pharmaceutically acceptable” indicates that the substance or composition must be compatible chemically and/or toxicologically, with the other ingredients comprising a formulation, and/or the mammal being treated therewith. Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals, and more particularly in humans.

In some embodiments, the pharmaceutical compositions further comprise one or more additional active ingredients. The additional active pharmaceutical ingredients can be, but are not limited to, an opiate, an analgesic, an NSAID, acetaminophen, an antipsychotic, a mood stabilizer, and an antidepressant.

A pharmaceutical composition containing a Ptched1 or Ptchd1 pathway antagonist and other therapeutic agents described herein (e.g., an opioid drug) can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

Any of the described Ptched1 or Ptchd1 pathway antagonist or pharmaceutical compositions identified herein can be formulated as a liquid formulation, as a solid formulation (including a powder or lyophilized formulation), or for aerosol administration. The compounds and pharmaceutical compositions can be formulated as a capsule or tablet, a time-release capsule or tablet, a powder, granules, a solution, a suspension in an aqueous liquid or non-aqueous liquid, an oil-in-water emulsion, or as a water-in-oil liquid emulsion. The compounds and pharmaceutical compositions can be formulated for oral administration, aerosol or inhalation administration, nasal administration, injection, infusion, topical administration, rectal administration, transmucosal administration, transdermal administration, intravenous administration, intradermal administration, subcutaneous administration, intramuscular administration, or intraperitoneal administration. In some embodiments, the pharmaceutical composition is administered parenterally.

Any of the compounds or pharmaceutical compositions identified herein can be formulated or packaged in single-dose or multi-dose format. In some embodiments, any of the compounds or pharmaceutical compositions identified herein can be formulated for repeat dosing.

The Ptched1 or Ptchd1 pathway antagonists for use in the described methods are administered to a subject in an amount that is sufficient to achieve the desired therapeutic effect in the subject. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors, including the activity of the particular Ptched1 or Ptchd1 pathway antagonist employed, the route of administration, the time of administration, or the rate of excretion of the particular compound being employed. Dosage can also depend on the duration of the treatment, or other drugs, compounds, and/or materials used in combination with the employed Ptched1 or Ptchd1 pathway antagonist. Age, gender, weight, condition, general health, and prior medical history of the subject being treated can also affect dosage. Methods for determining optimal dosages are described in the art. e.g., Remington: The Science and Practice of Pharmacy, Mack Publishing Co., 20th ed., 2000.

In some embodiments, kits containing one or more of the described Ptched1 or Pichd1 pathway antagonists or a pharmaceutical composition containing one or more Ptched1 or Ptchd1 pathway antagonists are described. In some embodiments, the kits comprise Ptched1 or Ptchd1 pathway antagonists or a pharmaceutical composition containing one or more Ptched1 or Ptchd1 pathway antagonists further comprise instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

In some embodiments, a kit further comprises two or more components, including at least one active pharmaceutical ingredient and one or more inactive ingredients, excipient, diluents, and the like, and optionally instructions for preparation of the dosage form by the patient or person administering the drug to the patient. In some embodiments, a kit may further comprise optional components that aid in the administration of the unit dose to a subject, including but not limited to: vials for reconstituting powder forms, syringes for injection, customized IV delivery systems, inhalers, etc. Additionally, a kit can contain instructions for preparation and administration of the compositions. The kit can be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

In some embodiments, a kit further includes an additional therapeutic agent. The additional therapeutic agent can be, but is not limited to, an opioid.

### C. Modified C. elegans for Analyzing Opioid Modulating Molecules

Described are genetically modified C. elegans animals expressing a mammalian MOR and having a loss of function mutation in the bgg10 gene. The modified C. elegans animals can be made using methods available in the art for genetically modifying C. elegans animals to insert a heterologous gene (e.g., mammalian or human MOR) or knockout an endogenous gene (e.g., bgg10). In some embodiments, the modified C. elegans animals are further modified to expresses a heterologous hPCTHD1 gene and/or a heterologous PTR-25 gene.

The modified C. elegans animals can be used to analyze the effectiveness of a compound in modulating opioid efficacy. The methods comprise contacting the modified C. elegans animal with the compound, or a plurality of compounds, and monitoring behavior of the animal in response to opioid. A decrease in movement of the modified C. elegans animal indicates increased effectiveness of the opioid. Movement of the modified C. elegans animal can be determined by methods available in the art for monitoring movement of C. elegans animals.

The modified C. elegans animals can be used screen for compounds that modulating opioid efficacy. The methods comprise contacting the modified C. elegans animal with one or more and monitoring behavior of the modified C. elegans animals in response to opioid. A decrease in movement of the animal indicates increased effectiveness of the opioid. Movement of the modified C. elegans animal can be determined by methods available in the art for monitoring movement of C. elegans animals

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Genetic Behavioral Screen for Modulators of Opioid Tolerance

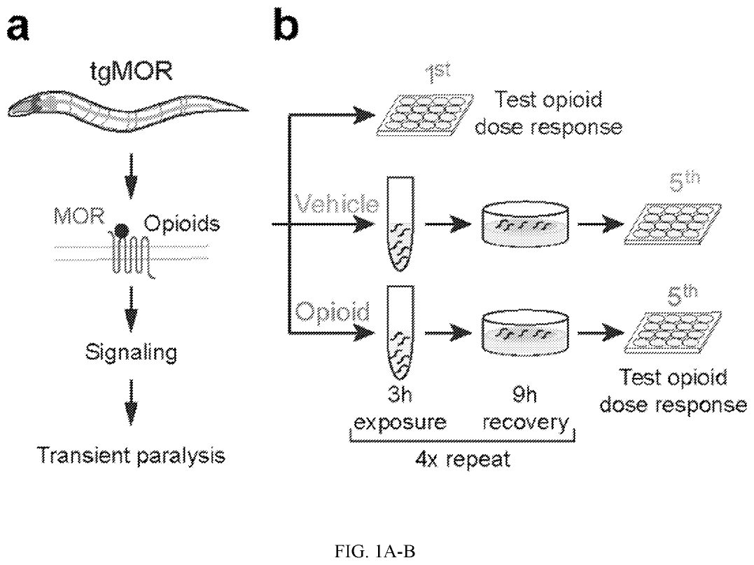

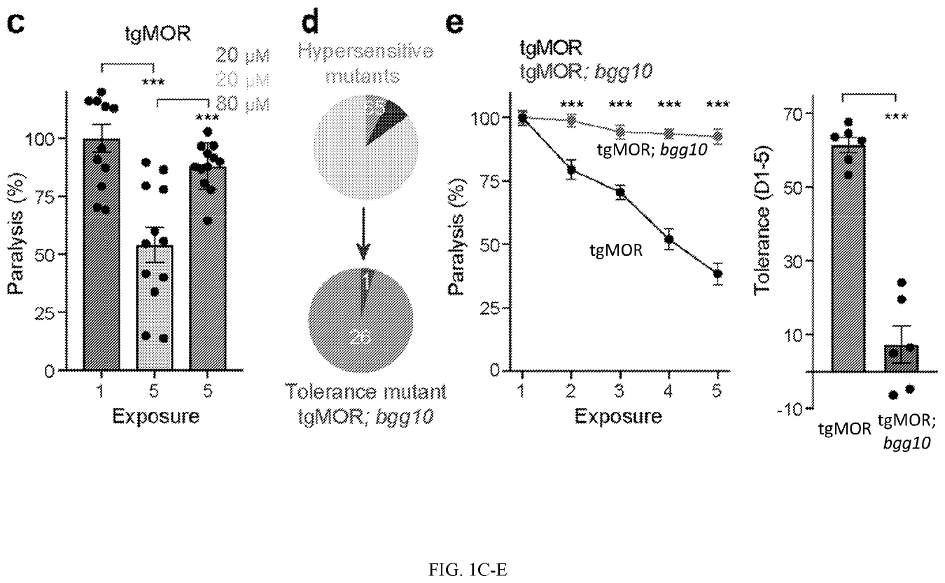

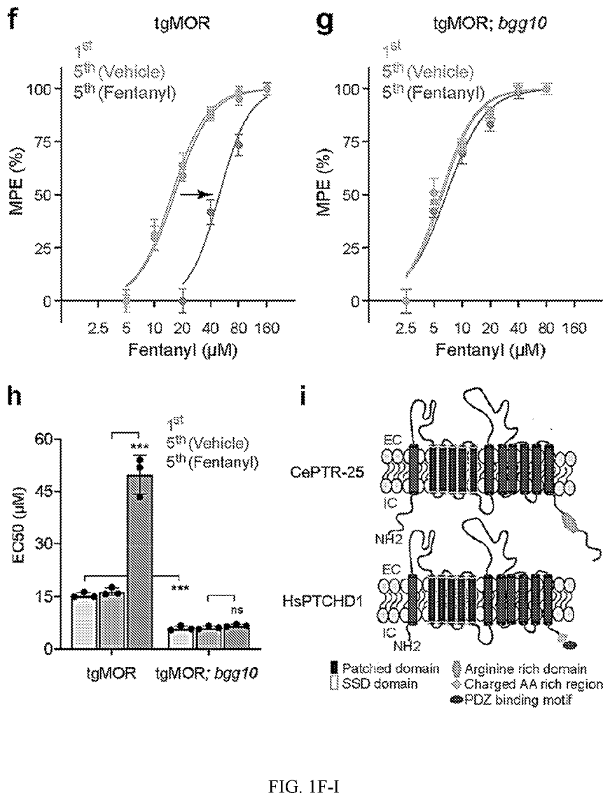

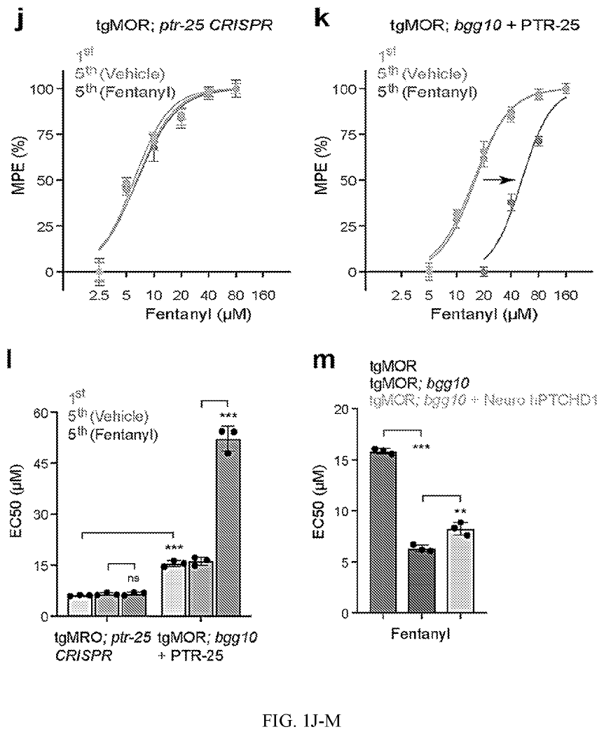

We recently engineered an in vivo whole animal model for studying opioid-induced behavior and signaling by transgenically expressing mammalian MOR (tgMOR) in the nervous system of C. elegans (Wang. D. et al. “Genetic behavioral screen identifies an orphan anti-opioid system.” Science, 2019 Sep. 20; 365 (6459): 1267-1273). Exposure of tgMOR animals to opioids suppresses their locomotion, which is used as a behavioral readout of opioid action (FIG. 1A). We leveraged this model to design a behavioral assay for opioid tolerance (FIG. 1B). When tgMOR worms were repeatedly exposed to the opioid fentanyl, the paralysis-inducing effect of the drug was markedly attenuated but restored by increasing the fentanyl concentration, the hallmark of behavioral tolerance (FIG. 1C). Dose response studies further confirmed induction of tolerance (FIG. 1F), evident from the increase in the half maximal effective concentration (EC₅₀) (FIG. 1F). Similar behavioral tolerance was observed with morphine.

To further validate the tgMOR platform for tolerance, we evaluated the behavior of an opioid hypersensitive mutant, tgMOR; rshp-1. Knockout of R7BP, the mammalian ortholog of RSBP-1, in mice produces opioid hypersensitivity and eliminates tolerance. tgMOR; rsbp-1 mutants failed to diminish responsiveness upon repeated exposure to fentanyl. Thus, a known tolerance regulator in mice is required for opioid tolerance in C. elegans. To test whether opioid tolerance is selective or generally observed in mutants with increased opioid sensitivity, we evaluated tgMOR; frpr-13 mutants for tolerance. FRPR-13 is a recently discovered conserved inhibitor of MOR signaling. In contrast to tgMOR; rsbp-1 animals, tgMOR; frpr-13 mutants had normal tolerance despite increased sensitivity to fentanyl. These observations indicate that opioid tolerance can be faithfully modeled in C. elegans and is governed by conserved genetic mechanisms.

Having established the validity of the tgMOR platform for studying opioid tolerance, we implemented a two-stage genetic behavioral screen to identify mutants with impaired tolerance. The first stage in this forward genetic approach isolated mutants with altered responses to opioids (as previously described: Wang D et al. 2019). In the second stage presented here, we screened a collection of hypersensitive tgMOR mutants for altered opioid tolerance based on prior evidence that tgMOR hypersensitive mutants affect MOR signaling (Wang D et al. 2019) and observations from rodents that loss of tolerance is accompanied by increased opioid responsiveness (Bohn L M et al. “Mu-opioid receptor desensitization by beta-arrestin-2 determines morphine tolerance but not dependence.” Nature, 2000 408:720-723 and Terzi D et al. “R7BP modulates opiate analgesia and tolerance but not withdrawal.” Neuropsychopharmacology, 2012 37:1005-1012). In total, we screened 27 hypersensitive tgMOR mutants and identified only one strain. tgMOR; bgg10, with impaired opioid tolerance (FIG. 1D). When compared to the parental tgMOR strain, tgMOR; bgg-10 mutants developed no observable tolerance to repeated fentanyl administration across 5 testing sessions (FIG. 1E). Dose-response studies showed a prominent right-ward shift in potency in tgMOR worms following chronic fentanyl treatment (FIG. 1F) and no shift in tgMOR; bgg10 mutants (FIG. 1G). This was quantitatively evaluated by showing that EC₅₀ increases in tgMOR animals but not tgMOR; bgg10 mutants (FIG. 1H). Collectively, these results demonstrate that tgMOR; bgg10 mutants have significantly reduced opioid tolerance.

Our analysis further confirmed that tgMOR; bgg10 mutants were hypersensitive and responded to opioids with faster paralysis. Further, bgg10 mutants were similar to non-transgenic, wild-type animals in failing to respond to opioids in the absence of tgMOR, indicating observed effects were specifically mediated by changes in MOR responses. Thus, deploying a two-stage unbiased, forward genetic screen led to the identification of a rare mutant with impaired behavioral tolerance to opioids.

### Example 2. PTR-25 Pichd1 Regulates Opioid Tolerance in C. elegans

Next, we sought to identify the genetic lesion in tgMOR; bgg10 that causes the opioid tolerance phenotype. To do so, we mapped mutations in tgMOR; bgg10 using whole genome sequencing, opioid phenotypic selection, and computational analysis of genome sequence data (Minevich G et al. “CloudMap: a cloud-based pipeline for analysis of mutant genome sequences.” Genetics, 2012 192:1249-1269). We found that tgMOR; bgg10) mutants harbored a predicted premature stop codon, Q1032stop (3094C>T) that is likely to generate a loss-of-function mutation in an unannotated gene F43D9.1. F43D9.1 encodes a protein with a Patched (Ptch) family domain and twelve-transmembrane topology. These are canonical features of the Ptch protein family that includes Patched, Dispatched. and numerous Patched-like proteins (Zhong Y et al. “Comprehensive analysis of patched domain-containing genes reveals a unique evolutionary pattern.” Genet Mol Res 2014 13:7318-7331). In C. elegans, F43D9.1 is part of a large, evolutionarily expanded group of Patched related family (PTR) proteins, prompting us to name this protein PTR-25. Our analysis indicates that Ptchd1 and Ptchd4 are the closest mammalian orthologs to PTR-25 (FIG. 1I). Like PTR-25, Ptchd1 and Ptchd4 contain a Ptch domain and a sterol sensing domain (SSD). Ptchd1 is expressed in the mammalian nervous system and implicated in neuropsychiatric conditions (Tora D. et al. “Cellular Functions of the Autism Risk Factor PTCHD1 in Mice.” J Neurosci (2017) 37:11993-12005; Wells M F et al. “Thalamic reticular impairment underlies attention deficit in Ptchd1(Y/−) mice.” Nature (2016) 532:58-63; and Noor A et al. “Disruption at the PTCHD1 Locus on Xp22.11 in Autism spectrum disorder and intellectual disability.” Sci Transl Med (2010) 2:49ra68), but relatively little is known about its function or mechanism of action.

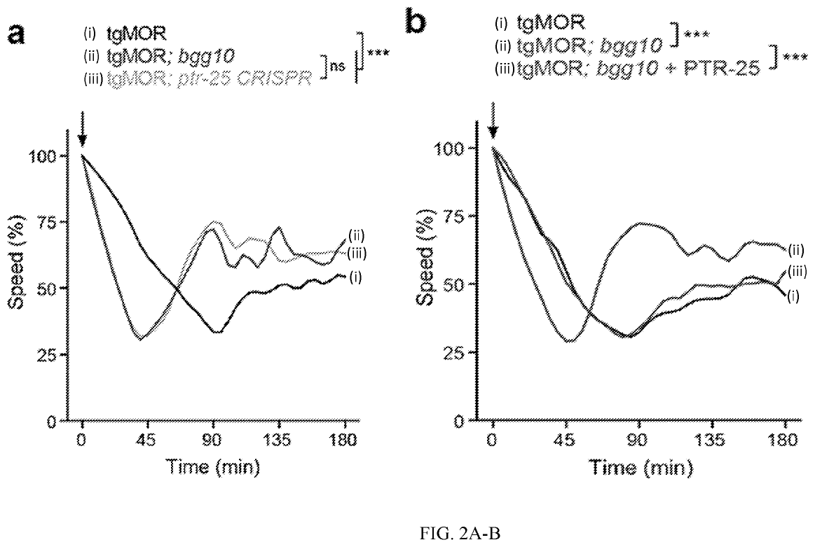

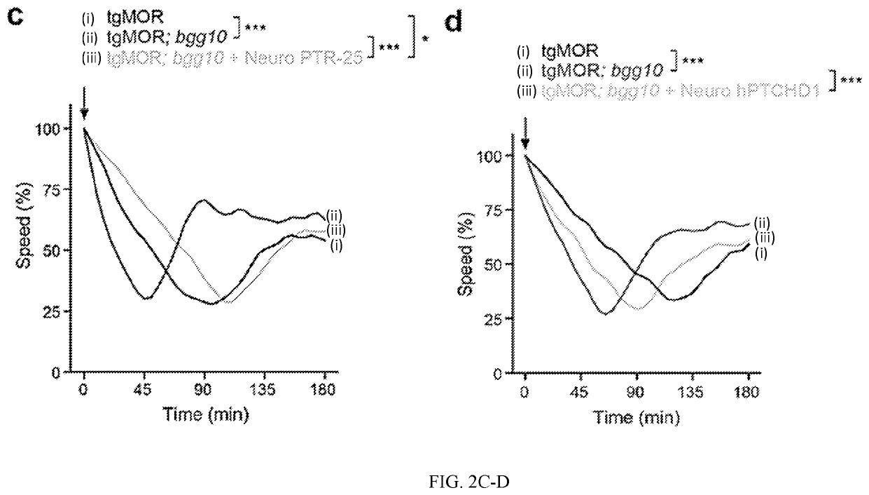

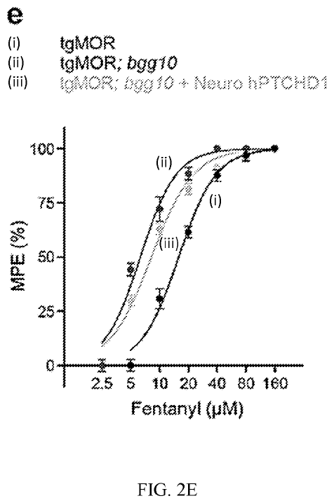

To confirm that the ptr-25 mutation affected opioid responses, we began by using CRISPR/Cas9 to edit the same premature stop codon, Q1032stop (3094C>T), present in tgMOR; bgg10) mutants into the parental tgMOR strain. This premature stop was predicted to truncate PTR-25 resulting in loss of the C-terminal intracellular portion of the receptor. We found that tgMOR; ptr-25 CRISPR animals exhibited greatly reduced opioid tolerance (FIG. 1J-L) and showed opioid hypersensitivity (FIG. 2A). As a second validation, we performed transgenic rescue experiments. A Mos single copy insertion (MosSCI) was used to express PTR-25 with its endogenous promoter in tgMOR; bgg10 mutants. This completely restored behavioral tolerance to fentanyl (FIG. 1K-1L) and opioid sensitivity (FIG. 2B). Transgenic MosSCI expression of PTR-25 using a pan-neuronal promoter also rescued opioid hypersensitivity of tgMOR; ptr-25 (hgg10) animals (FIG. 2C). Collectively, these results indicate that PTR-25 is the causal gene responsible for loss of opioid tolerance and hypersensitivity in tgMOR; bgg10) mutants.

Finally, we tested whether Ptchd1 affects MOR-mediated opioid responses by transgenically expressing human PTCHD1 in the nervous system of tgMOR; bgg10 animals. We observed a significant rescue of hypersensitivity to fentanyl in these animals with EC₅₀) increasing compared to tgMOR; bgg10) animals (FIG. 1M); i.e., tgMOR; bgg10) animals expressing PTCHD1 behaved more like tgMOR animals. Moreover, response times to paralysis with fentanyl were longer in tgMOR; bgg10) mutants expressing human PTCHD1 compared to tgMOR; bgg10 animals and dose responses were shifted (FIG. 2D-E). In summary, our results provide in vivo genetic evidence that PTR-25 and Ptchd1 are functional orthologs that regulate opioid tolerance and responsiveness.

The tgMOR; bgg10, tgMOR; bgg10)-PTR-25, tgMOR; bgg10); hPTCHD1, and tgMOR; bgg10)+neuro hRTCHD1 C. elegans animals can be used to screen for drugs that affect opioid efficacy. The effectiveness of a compound in modulating opioid efficacy can be analyzed by contacting the C. elegans animal with the compound and monitoring behavior of the animal in response to opioid, wherein decreased movement indicates increased effectiveness of the opioid. Efficacy of the opioid can be analyzed with respect to dosage required to produce a desired result, duration of response, or response to repeat administration of the opioid.

### Example 3. Deletion of Ptchd1 in Mice Enhances Opioid Efficacy and Eliminates Tolerance

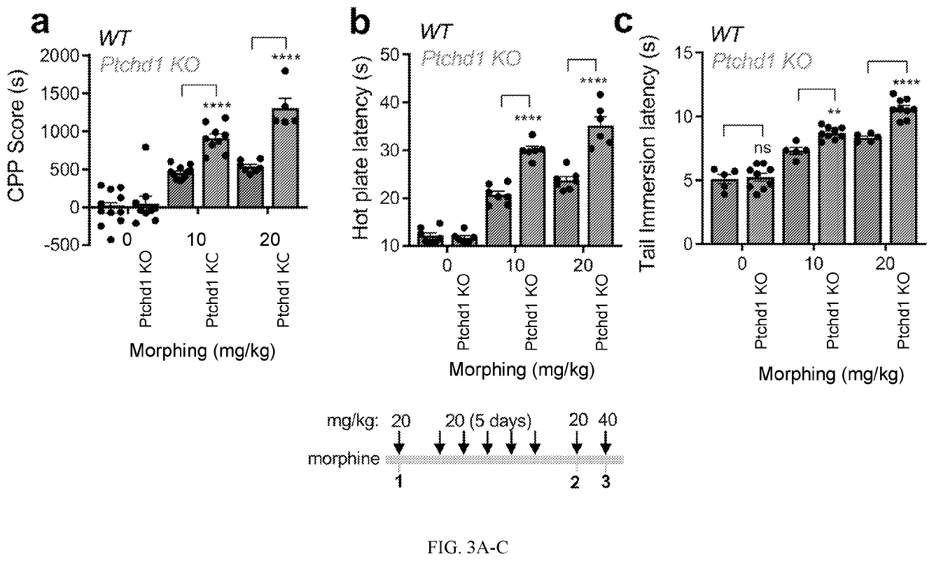

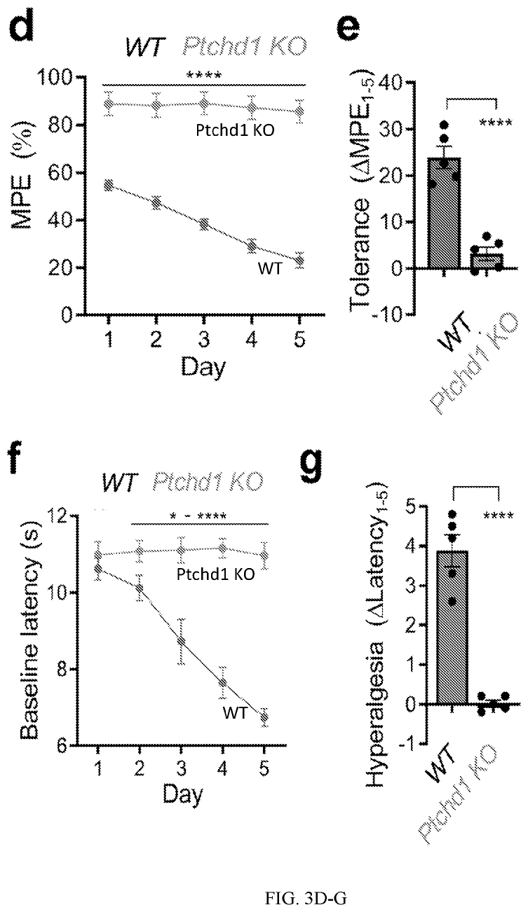

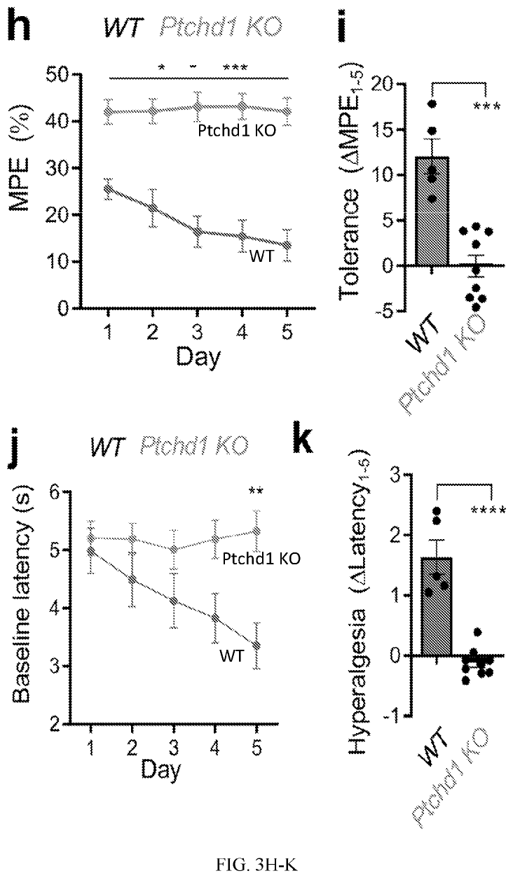

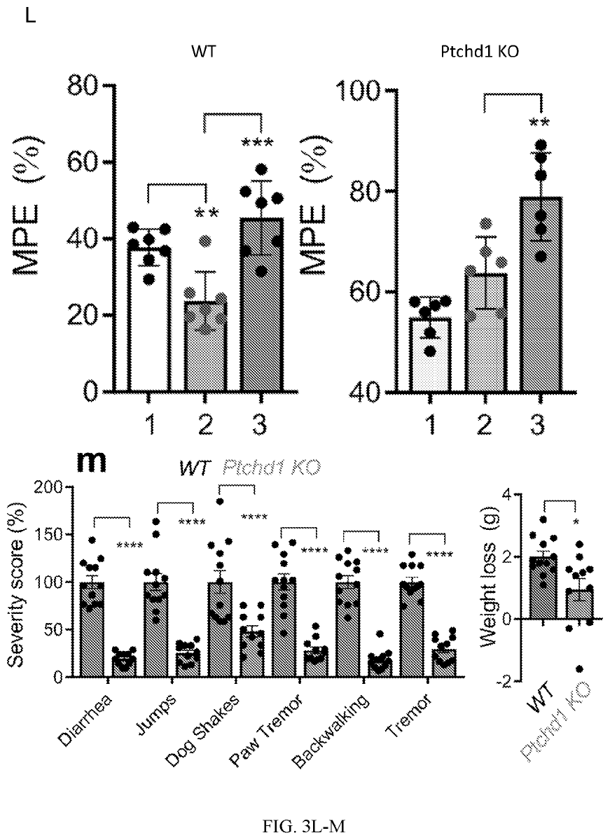

To test the role of Ptchd1 in regulating opioid responses in mammals and to probe its translational relevance, we evaluated mice with a disruption in Ptchd1 (Ptchd1 KO). Because a previous report indicated that Ptchd1 KO mice are hyperactive (Wells M F et al. 2016), we evaluated their opioid responses in behavioral assays that are insensitive to changes in activity levels. First, we used the conditioned place preference (CPP) assay, which evaluates the reinforcing properties of opioids by measuring relative amount of time spent in a drug-paired chamber as compared to an unpaired chamber. Indeed, at the no-drug baseline, both Ptchd1 KO mice and their wild-type (WT) littermates behaved equally (FIG. 3A). When injected with morphine systemically, Ptchd1 KO mice exhibited substantially higher preference for the drug-paired chamber indicating they have an augmented response to the rewarding effects of morphine (FIG. 3A).

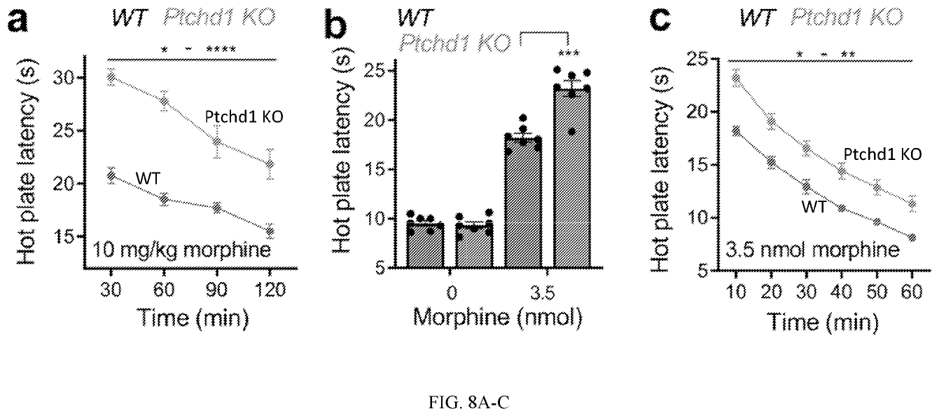

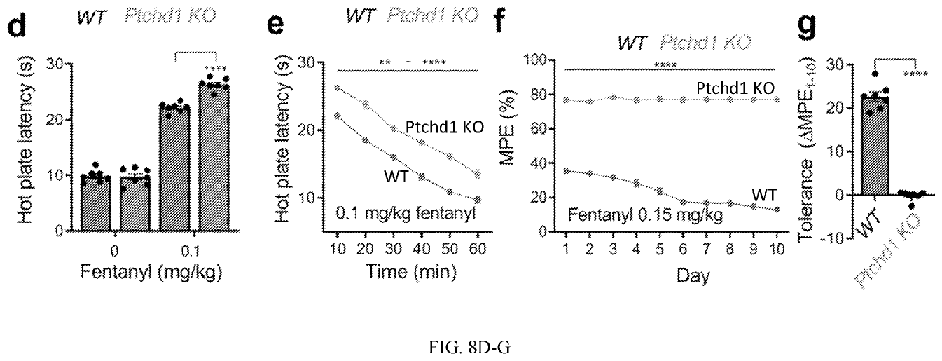

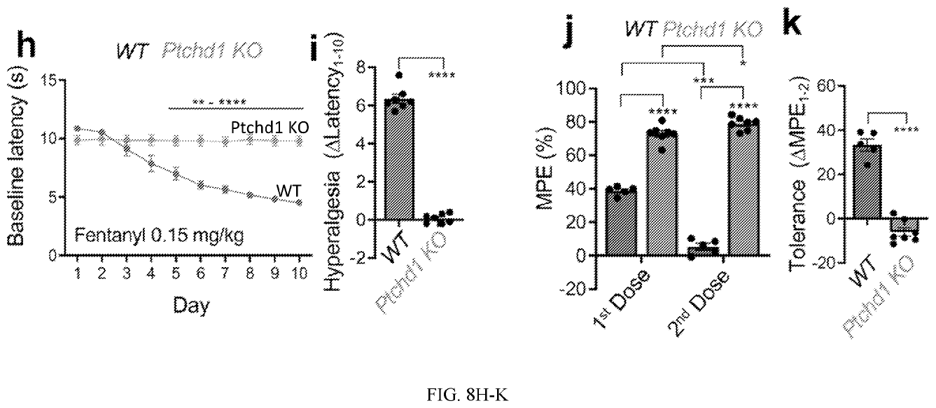

Next, we tested nocifensive behavior of mice in pain paradigms. We found that baseline nociceptive thresholds of Ptchd1 KO animals were similar to WT littermates in both hot plate and tail immersion assays (FIG. 3B-C). However, morphine produced a greater analgesic response in Ptchd1 KO mice relative to WT controls in both paradigms when injected systemically (FIG. 3B-C; FIG. 8A). Morphine also produced greater analgesia in Ptchd1 KO mice when injected directly into the brain (FIG. 8B-C). Furthermore, Ptchd1 KO mice exhibited similarly enhanced analgesia in response to fentanyl (FIG. 8D-E). These data indicate that Ptchd1 restricts the analgesic efficacy of opioids in mice.

We next assessed tolerance by subjecting mice to repeated daily opioid injections. When injected with morphine, WT mice displayed a substantial reduction in opioid analgesia in the hot plate assay with each subsequent morphine application (FIG. 3D-E). This was accompanied by the development of hyperalgesia as evidenced by progressive reduction in baseline latency (FIG. 3F-G). In contrast, this chronic treatment regimen in Ptchd1 KO mice failed to diminish responsiveness suggesting chronic tolerance did not occur (FIG. 3D-E). Ptchd1 KO mice also did not develop morphine induced hyperalgesia (FIG. 3F-G). We then doubled our chronic tolerance induction window and used fentanyl. Again, we found that Ptchd1 KO mice did not develop any signs of tolerance even in this extended chronic paradigm with a stronger opioid analgesic. Pichd1 KO mice also did not develop opioid induced hyperalgesia in this paradigm (FIG. 8H-I).

We further evaluated Ptchd1 KO mice using an acute tolerance paradigm where morphine injection was repeated immediately upon waning of the initial analgesic response. While WT mice completely lost their morphine responsiveness to repeated doses, there was no sign of acute tolerance in Ptchd1 KO (FIG. 8J-K).

Our observations using the tail immersion assay, which principally relies upon spinal reflexes, largely paralleled our findings with the hot plate assay. Once again, we observed that Ptchd1 KO mice did not develop tolerance or hyperalgesia (FIG. 3H-K). Dose-escalation studies showed that increasing morphine restored the loss of efficacy in WT mice, and augmented analgesia in Ptchd1 KO animals confirming tolerance was abrogated (FIG. 3L). In fact, the responses of Ptchd1 KO mice were sometimes sensitized following opioid exposure (FIG. 8J).

We evaluated how loss of Ptchd1 affected dependence following chronic opioid exposure. Strikingly, Ptchd1 KO mice had greatly reduced somatic withdrawal signs and showed significantly less body weight loss following chronic morphine treatment (FIG. 3M). Thus, reduced withdrawal response accompanies impaired tolerance in Ptchd1 KO animals. Taken as a whole, our observations demonstrate that Ptchd1 negatively regulates behavioral responses to opioids and development of tolerance in mice, similar to its ortholog PTR-25 in C. elegans.

### Example 4. Ptchd1 Controls MOR-Mediated Regulation of Neuronal Activity

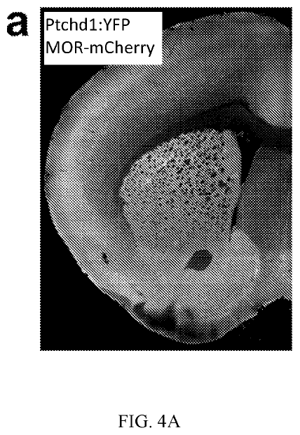

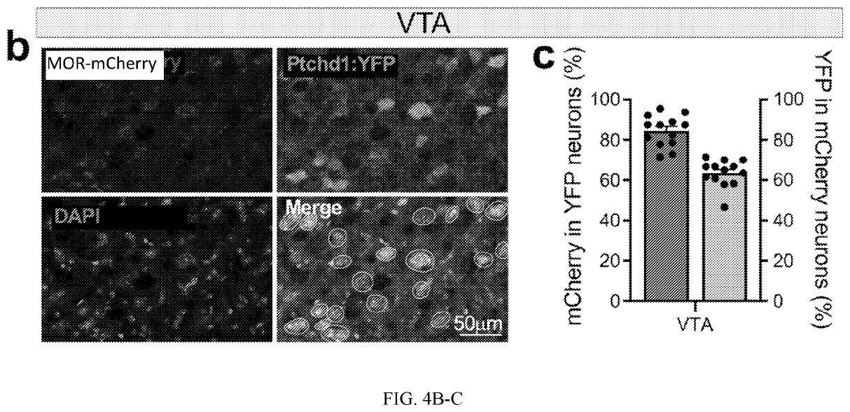

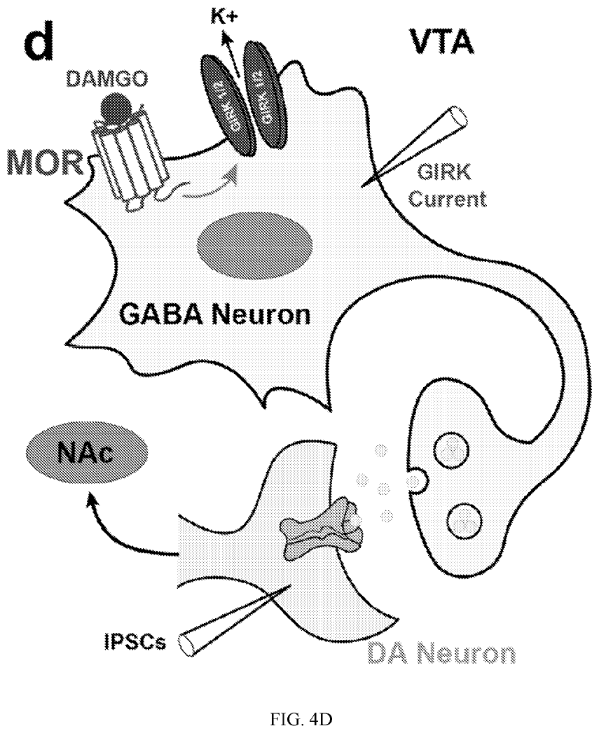

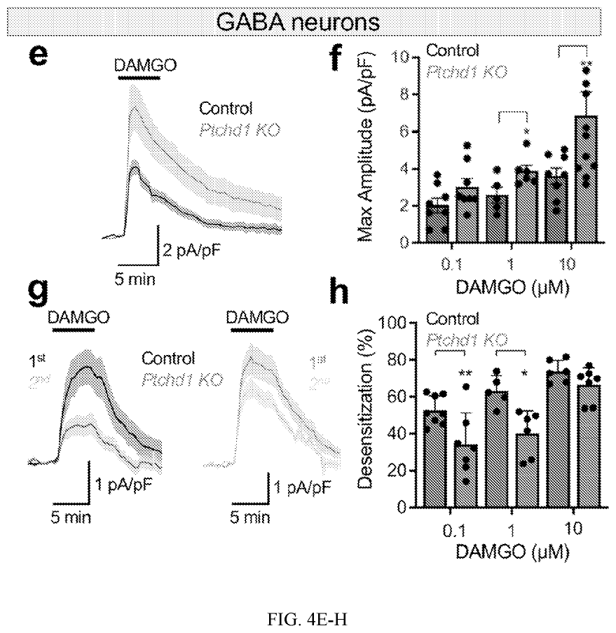

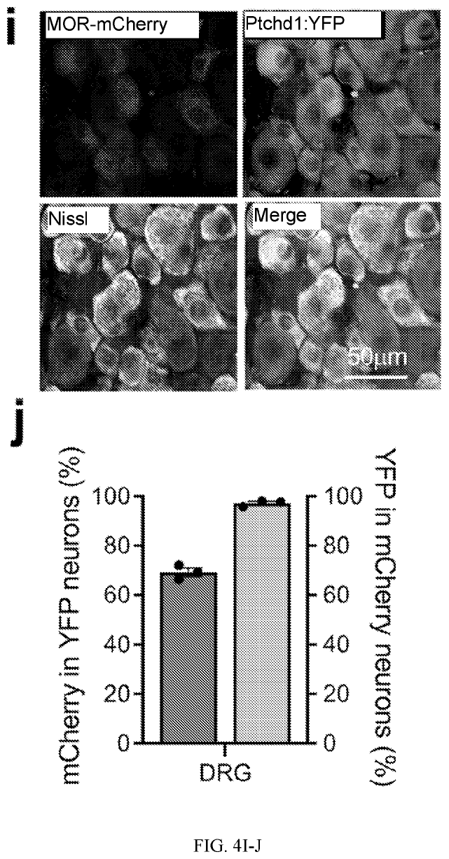

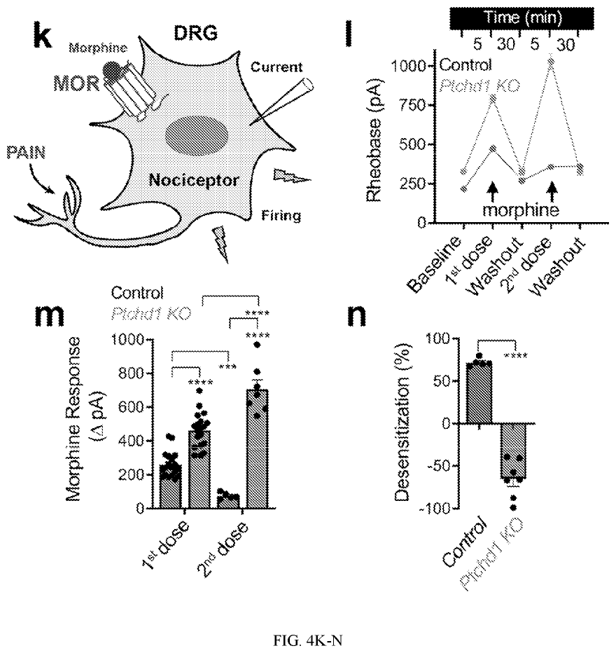

To determine how loss of Ptchd1 affects behavioral responses to opioids, we examined the relationship between Ptchd1 and MOR in mouse neural circuits using slice electrophysiology. To identify neurons expressing Ptchd1 and MOR, we crossed Ptchd1 KO mice expressing YFP from a targeted allele with knock-in MOR-mCherry reporter mice. Consistent with previous reports, we found Ptchd1 expression in several regions of the central nervous system. This included prominent expression in areas associated with reward and opioid actions including nucleus accumbens (NAc), ventral tegmental area (VTA), thalamus and locus coeruleus (FIG. 4A-B). Ptchd1 was co-expressed with MOR in many of these brain regions (FIG. 4A-B). Furthermore, we observed Ptchd1 expression in peripheral dorsal root ganglia (DRG) neurons where opioids can also act (FIG. 4I-J). Co-expression was particularly prominent in the VTA and in the DRGs where the majority of MOR-expressing neurons expressed Ptchd1 (FIG. 4C and 4I). We further confirmed co-expression of endogenous MOR and Ptchd1 mRNA using in situ hybridization. This pattern of Ptchd1 expression in mice was consistent with its broad presence across the human nervous system.

We further examined brain region size, cell density and neuron size in Ptchd1 KO mice. Our results indicate that Ptchd1 elimination had no or minimal effects on the size of the VTA or NAc. and cell density in these brain regions. Neuron size was also unaltered in the VTA and NAc, while a very small but significant increase was detected in the size of DRG neurons. Overall, our results are consistent with previous morphological findings (Tora D. et al. 2017 and Ung D C et al. “Ptchd1 deficiency induces excitatory synaptic and cognitive dysfunctions in mouse.” Mol Psychiatry (2018) 23:1356-1367).

To dissect the cellular mechanisms of Ptchd1 action, we first focused on the VTA neurons because the effects of opioids in this brain region are well understood. Here, MOR is predominantly expressed by the GABAergic interneurons where it activates G protein inwardly rectifying K+ (GIRK) channels reducing inhibitory output (FIG. 4D). Application of the MOR agonist DAMGO elicited GIRK-mediated inward currents, which were significantly augmented upon loss of Ptchd1 (FIG. 4E-F). Repeated application of DAMGO after washout resulted in smaller GIRK currents relative to the first exposure due to receptor desensitization (FIG. 4G-H), which is thought to be a cellular correlate of tolerance. Importantly, less desensitization occurred in Ptchd1 KO neurons (FIG. 4G-H). Dose-response studies confirmed these observations and additionally revealed that loss of Ptchd1 preferentially augmented opioid efficacy, as the deletion of Ptchd1 had a larger effect on MOR-mediated GIRK regulation with increasing DAMGO concentration (FIG. 4F). In contrast, loss of desensitization was more prominent upon low level of MOR stimulation (FIG. 4H).

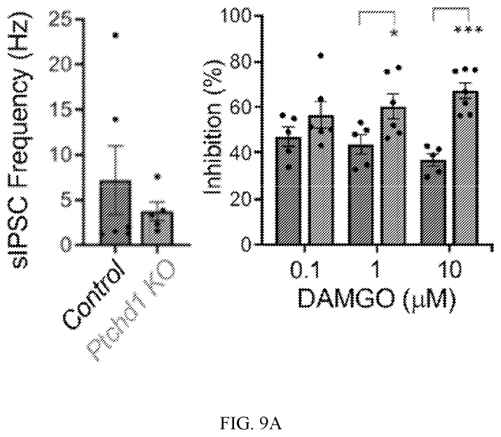

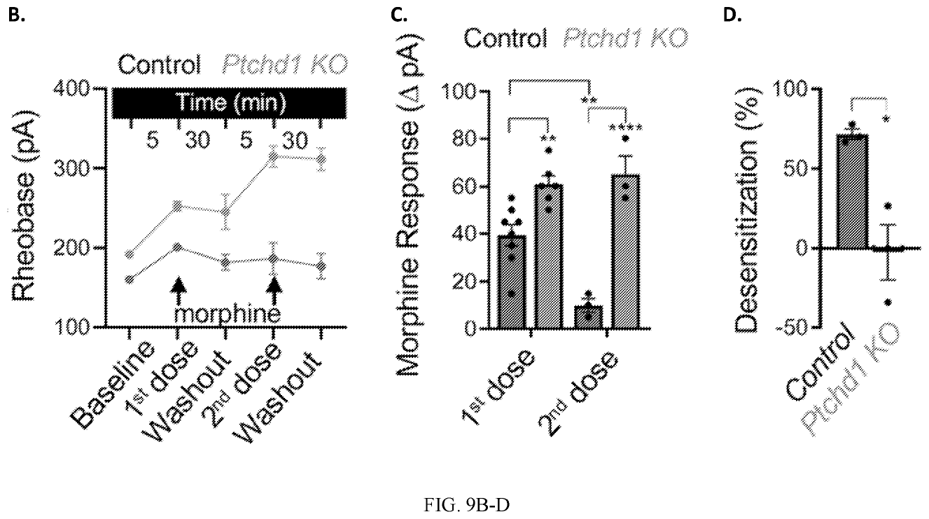

The increased MOR activation in GABAergic neurons in Ptchd1 KO would be expected to reduce their inhibitory influence over dopamine (DA)-releasing neurons. DA neurons in the VTA directly encode reward through changes in dopamine release, which is thought to be responsible for the reinforcing effects of opioids (FIG. 4D). Therefore, we next measured opioid modulation of GABAergic inputs into DA neurons by recording Inhibitory Post Synaptic Currents (IPSC). Application of DAMGO reduced IPSC frequency to a greater extent in the Ptchd1 KO compared to WT slice. Challenging the same preparation with an escalating concentration of DAMGO did not augment the responses in WT neurons, likely due to MOR being fully desensitized. In contrast, we observed increased inhibition in the Ptchd1 KO with increasing DAMGO concentrations, which is consistent with MOR-desensitization being impaired (FIG. 9A).

To explore how general the role of Ptchd1 is in the brain, we next recorded from striatal medium spiny neurons (MSN) of the NAc, a neuronal population relevant for opioid actions where Ptchd1 and MOR are co-expressed. In these experiments, we used morphine to further probe agonist specificity of Ptchd1 effects. Similar to the VTA, we observed larger opioid responsiveness of MSNs lacking Ptchd1 (FIG. 9B). Upon repeated morphine challenge, we observed pronounced desensitization in control neurons which did not occur in Pichd1 KO neurons (FIG. 9C-D).

Finally, we turned our attention to DRGs which co-express MOR and Ptchd1 (FIG. 4I-J), and are a major peripheral site for MOR-mediated effects on analgesia and tolerance. We relied on a genetic labeling strategy coupled with electrophysiological profiling to record specifically from the nociceptors that are positive for MOR and Ptchd1 expression (FIG. 4K). Similar to the central nervous system, we found that opioid-mediated suppression of excitability was significantly more pronounced in Ptchd1 KO nociceptors relative to controls (FIG. 4L-M). Strikingly, while control neurons substantially desensitized upon repeated application of morphine, we observed that excitability of Ptchd1 KO nociceptors was suppressed even more prominently upon morphine re-exposure. Thus, instead of desensitization. DRG nociceptors from Ptchd1 KO animals increase their sensitivity to morphine which parallels behavioral sensitization of Pichd1 KO in the tail immersion paradigm.

Based on these electrophysiological experiments, we conclude that Ptchd1 acts in the same neuronal populations as MOR across the nervous system to suppress opioid modulation of neuronal activity and to promote desensitization of MOR responses.

### Example 5. Ptchd1 Controls MOR Trafficking and Surface Abundance

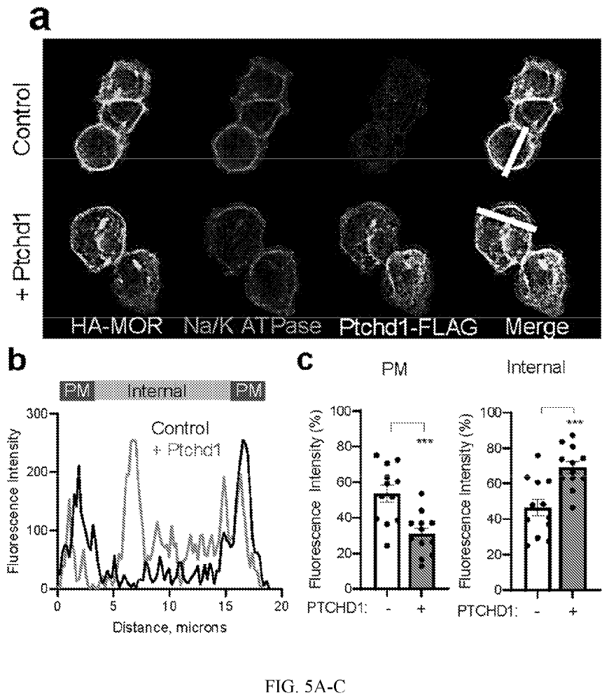

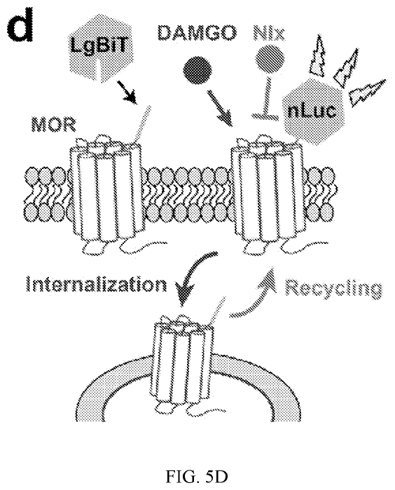

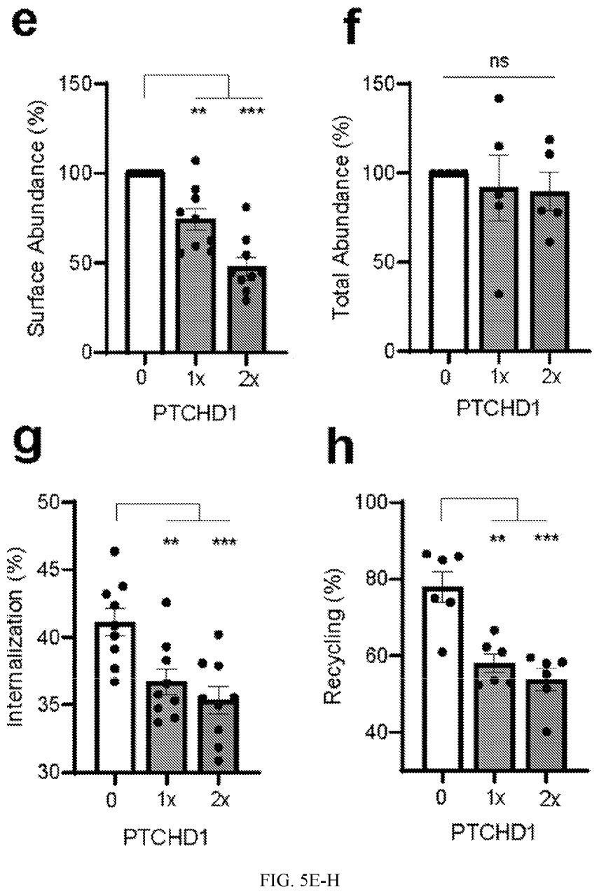

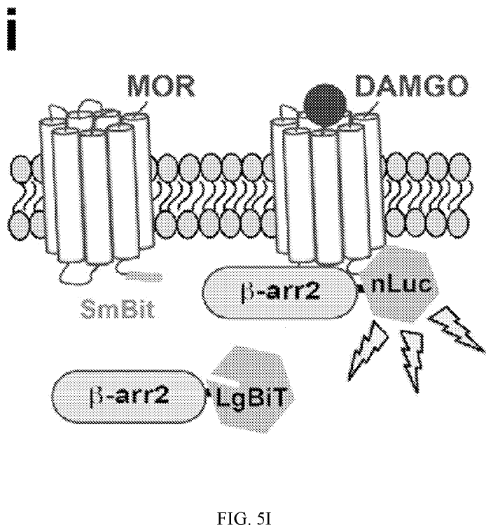

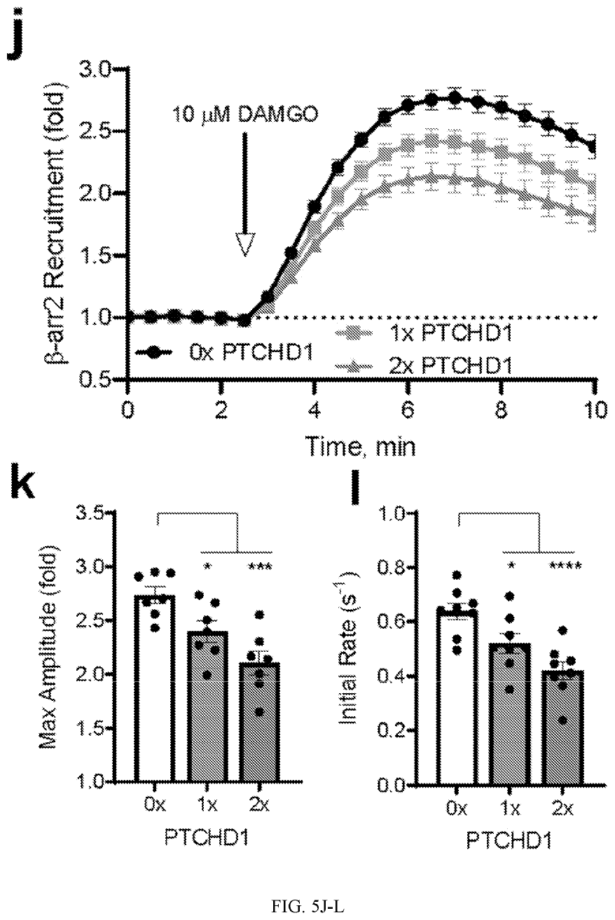

We next set out to determine the molecular mechanism by which Ptchd1 influences MOR function. Given the increased efficacy of opioid responses at the behavioral and cellular levels in Ptchd1 KO animals, we examined whether Ptchd1 affects MOR expression and localization to impact its signaling strength. For these studies, we turned to cell-based assays in HEK293 cells reconstituted with MOR. When expressed alone, MOR was robustly localized on the plasma membrane (FIG. 5A-C). In contrast, introduction of Ptchd1 promoted intracellular retention of MOR and reduced its targeting to the plasma membrane (FIG. 5A-C). To examine MOR localization dynamics, we employed a nanoluciferase complementation system (FIG. 5D). Again, over-expression of Ptchd1 decreased the surface abundance of MOR (FIG. 5E) without affecting overall MOR expression (FIG. 5F). These results indicate that Ptchd1 is sufficient for regulating MOR trafficking under basal conditions.

Next, we studied how Ptchd1 affects trafficking of MOR following activation by opioids. We found that co-expression of Ptchd1 significantly inhibited DAMGO-induced MOR internalization (FIG. 5G). Next, the MOR antagonist naloxone was added to examine the recovery of MOR back to the cell surface. We observed MOR recycling that was significantly inhibited by expressing Ptchd1 (FIG. 5H). Interestingly, overexpression of C. elegans PTR-25 similarly impaired MOR internalization triggered by DAMGO and produced an even stronger effect than human Ptchd1. This effect was specific to MOR, as Ptchd1 overexpression did not affect internalization of another GPCR, the B2 adrenergic receptor. Thus. Ptchd1 and PTR-25 are general inhibitors of MOR trafficking both to and from the plasma membrane.

Previous studies established β-arrestins as key players that control trafficking of many GPCRs including MOR. Therefore, we next examined the effects of Ptchd1 on the association of MOR with β-arrestin2 (FIG. 5I). Consistent with Ptchd1 inhibiting MOR internalization, we found that Ptchd1 expression inhibited agonist induced β-arrestin2 recruitment (FIG. 5J-L). We did not observe this influence with V2 vasopressin receptor, another GPCR prominently regulated by β-arrestin2. Overall, these results support the model that Ptchd1 regulates MOR trafficking by affecting its association with regulatory adaptors.

### Example 6. Cholesterol Mediates Effects of Pichd1 on MOR Trafficking

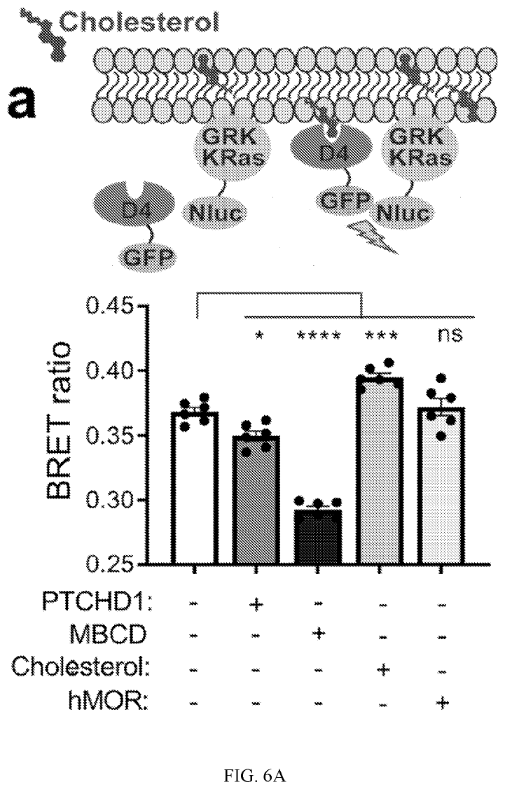

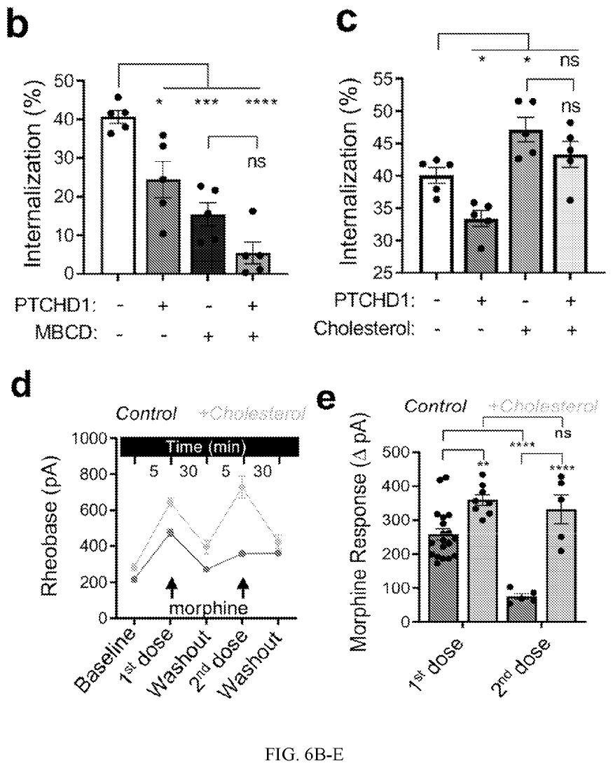

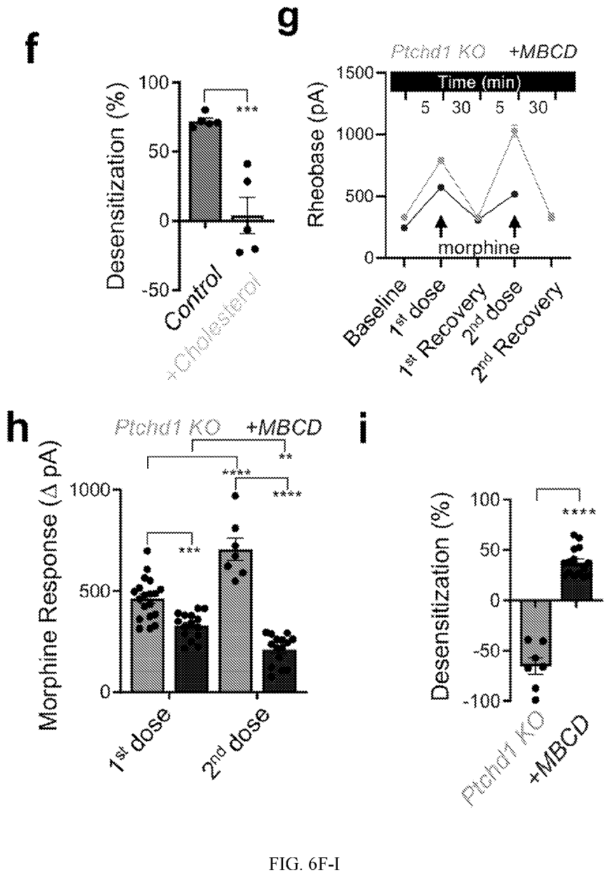

Prior studies have shown that several Patched family members regulate cholesterol transport. Therefore, we tested whether Ptchd1 regulates cholesterol levels to affect MOR trafficking. Using a molecular biosensor approach, we found that overexpression of Ptchd1 significantly decreased the cholesterol content in the plasma membrane (FIG. 6A). This effect was similar to the action of the well-known cholesterol-depleting drug methyl-β-cyclodextrin (MBCD) and was the opposite from direct cholesterol supplementation. We tested another multi-pass transmembrane protein, MOR, as a control and did not observe effects on membrane cholesterol levels (FIG. 6A).

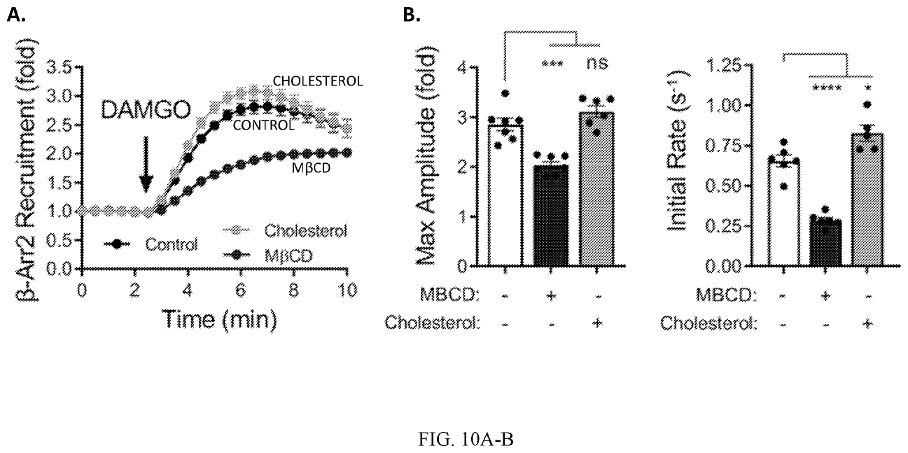

We next determined whether Ptchd1 modulation of cholesterol affected MOR trafficking. Consistent with prior observations, depleting cholesterol with MBCD inhibited MOR internalization (FIG. 6B) whereas cholesterol addition had the opposite effect (FIG. 6C). Accordingly, increasing or reducing cholesterol levels had opposing effects on β-arrestin recruitment to MOR (FIG. 10A-B). Importantly, both depletion and enrichment of cholesterol enhanced and occluded, respectively, the effects of Ptchd1 overexpression on MOR internalization (FIG. 6B-C).

To examine an endogenous neuronal setting, we turned to acute cultures of adult DRG nociceptor neurons. First, we incubated genetic control DRG cultures with cholesterol examining the impact on opioid-mediated suppression of excitability (FIG. 6D). We found that cholesterol treatment increased opioid inhibition of DRG firing (FIG. 6E-F). Remarkably, after the first application of morphine and subsequent washout, a repeated challenge with morphine resulted in the same degree of excitability decrease. Thus, cholesterol supplementation prevented response desensitization to morphine. To test the role of Ptchd1, we performed similar experiments using DRGs from Ptchd1 KO animals. We observed that enhanced excitability of Pichd1 KO cells was reversed by treatment with MBCD (FIG. 6G-H). Furthermore, cholesterol depletion with MDCB restored response desensitization of Ptchd1 KO neurons to repeated morphine application (FIG. 6H-I). Collectively, these findings with both cell-based approaches and endogenous DRGs support the model that Ptchd1 regulates MOR desensitization by affecting cholesterol availability.

### Example 7. Discussion

In examples 1-6, we reported the identification of a novel regulator and mechanism controlling opioid tolerance. Chronic stimulation of MOR and other GPCRs triggers desensitization which leads to behavioral tolerance. A canonical example of tolerance, with particularly prominent clinical implications, is loss of therapeutic efficacy with prolonged use of opioids. Tolerance diminishes both the analgesic and euphoric effects of opioid drugs, necessitating dose escalation. This in turn contributes to problematic side effects, dependence and overdose fatalities.

Understanding the mechanisms underlying opioid tolerance and GPCR desensitization has been a major research focus for decades. Here, we applied unbiased forward genetics to the study of opioid tolerance for the first time. Our in vivo behavioral approach identified PTR-25/Ptchd1 as an evolutionarily conserved regulator of opioid tolerance. These findings introduce a new molecular target for intervention to combat tolerance, but not reward-associated dependence, which remains the most significant limitation of opioid therapies.

Because opioid tolerance is a behavioral phenomenon, genetic screens for tolerance require an in vivo setting, such as the animal model we deployed. Our study further leveraged the power of C. elegans in addressing important biomedical questions, which now includes drug tolerance.

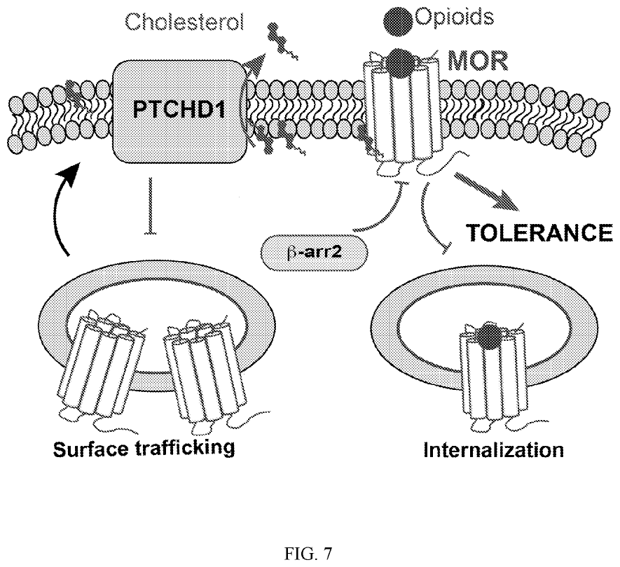

We have shown that Ptchd1/PTR-25 acts to regulate MOR trafficking to enable behavioral tolerance. Our findings support a model in which Ptchd1 regulates MOR trafficking by changing the membrane lipid environment via effects on cholesterol (FIG. 7). Our results indicate that Ptchd1 regulates MOR internalization and inhibits its interaction with β-arrestin. This suggests that Ptchd1 restricts MOR endocytosis allowing prolonged signaling from the cell surface possibly reducing MOR re-sensitizing internalization and recycling. The results indicate that Ptchd1 facilitates persistent MOR signaling from the plasma membrane that ultimately induces tolerance. The data also indicate Ptchd1 links behavioral tolerance and acute MOR desensitization, as our findings indicate that loss of Ptchd1 makes neurons more resistant to the desensitizing effects of opioids.

Interestingly, we found that Pichd1 KO mice also exhibited less severe somatic withdrawal signs despite their augmented opioid sensitivity.

On a cellular level, our results indicate that Ptchd1 regulates MOR trafficking via effects on cholesterol. Cholesterol can affect GPCRs by direct binding and indirectly by regulating receptor dynamics in the lipid environment. Structural studies have shown that MOR possesses a cholesterol binding pocket. Cholesterol has also been shown to regulate trafficking and signaling of MOR (Qiu Y et al. “Cholesterol regulates micro-opioid receptor-induced beta-arrestin 2 translocation to membrane lipid rafts.: Mol Pharmacol (2011) 80:210-218 and Zheng H. et al. “Palmitoylation and membrane cholesterol stabilize mu-opioid receptor homodimerization and G protein coupling.” BMC Cell Biol (2012) 13:6. We have shown that Ptchd1 reduces the cholesterol content in the plasma membrane, and that its effects on MOR trafficking depend on cholesterol availability.

In addition to affecting receptor desensitization and tolerance, the data indicate that Ptchd1 influences the levels of MOR on the cell surface. Overexpression of Ptchd1 led to intracellular sequestration of MOR. In contrast, loss of Ptchd1 substantially increased the efficacy of opioid signaling in neurons. Ptchd1 appears to have impeded forward targeting of MOR to the plasma membrane thereby serving as a general inhibitor of MOR trafficking.

### Example 8. Manipulating HDL/LDL Cholesterol in Inhibiting Opioid Tolerance and Decreasing Withdrawal Symptoms

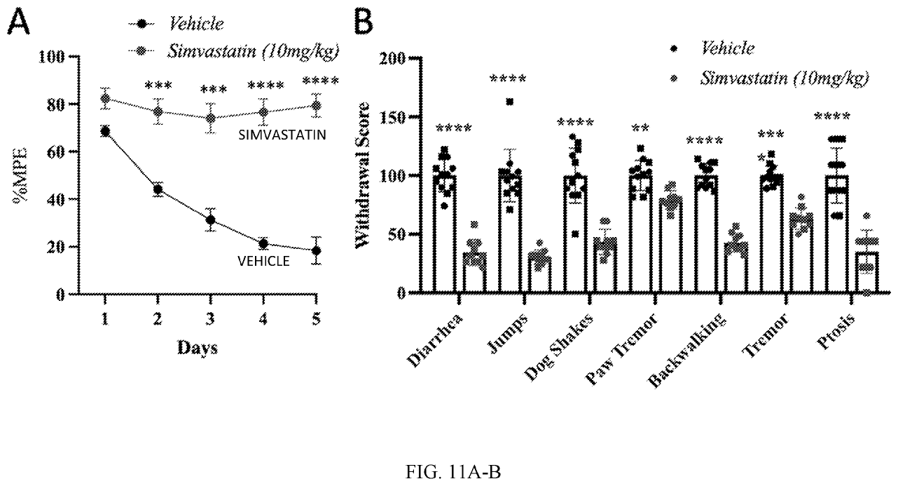

To test effectiveness and utility of manipulations with HDL/LDL cholesterol experimentally, we evaluated the effect of treating mice with simvastatin (10 mg/kg for 6 days) on morphine tolerance and withdrawal. We found that this drug treatment completely prevented the development of analgesic tolerance (FIG. 11A). We further tested the effects on the somatic withdrawal by continuing these mice on escalating regime of morphine injections: 40, 60, 80 and 100 mg/kg administered each daily. On testing day of 100 mg/kg morphine, animals were injected with naloxone (1 mg/kg; i.p.) 3 hours after morphine injection and evaluated for withdrawal signs. The results showed substantially diminished somatic withdrawal signs in mice treated with simvastatin (FIG. 11B). Together, these results support the idea that manipulating with cholesterol to increase its availability (higher HDL/LDL ratio) produces beneficial effects.

### Example 9. CRISPR/Cas9-Mediated Suppression of Ptchd1 Expression

CRISPR/Cas9 gene editing was used to alter the function of the Pthcd1 ortholog, ptr-25, in C. elegans (FIG. 1), tgMOR animals where CRISPR editing was used to impair ptr-25 (tgMOR; ptr-25 CRISPR) exhibited decreased opioid tolerance (FIGS. 1J and L). A CRISPR/Cas9 system targeting the Ptch1 gene can also be used in mammals, including humans. An exemplary target for CRISPR-mediated modulation of human PTCHD2 comprises SEQ ID NO: 2.

SEQ ID NO: 2
GATTGGGTTTTACATATATGAGTCTATAGAATACTGGAACACTAGTGTCCAAGAAGATGTTCTAGAATACACCAA
GGGGTTTGTGCGGATATCCTGGTTTGAGAGCTATTTAAATTACCTTCGGAAACTCAATGTATCCACTGGCTTGCC
TAAGAAAAATTTCACAGACATGTTGAGGAATTCCTTTCTGAAAGCCCCTCAATTTTCACATTTTCAAGAGGACAT
CATCTTCTCTAAAAAATACAATGATGAGGTCGATGTAGTGGCCTCCAGAATGTTTTTGGTGGCCAAGACCATGGA
AACAAACAGAGAAGAACTCTATGATCTCTTGGAAACCCTGAGGAGACTTTCTGTCACCTCCAAGGTGAAGTTCAT
CGTCTTCAATCCGTCCTTTGTATACATGGATCGATATGCCTCCTCTCTGGGAGCCCCCCTGCACAACTCCTGCAT
CAGTGCTTTGTTCCTGCTCTTCTTCTCGGCATTCCTGGTGGCAGATTCACTGATTAACGTCTGGATCACTCTCAC
AGTTGTGTCCGTGGAGTTTGGAGTGATAGGTTTCATGACATTATGGAAAGTAGAACTGGACTGCATTTCTGTGCT
ATGCTTAATTTATGGAATTAATTACACAATTGACAATTGTGCTCCAATGTTATCCACATTTGTTCTGGGCAAGGA
TTTCACAAGAACTAAATGGGTAAAAAATGCCCTGGAAGTGCATGGGGTAGCTATTTTACAGAGTTACCTCTGCTA
TATTGTTGGTCTGATTCCTCTTGCAGCTGTGCCTTCAAATCTGACCTGTACACTGTTCAGGTGCTTGTTTTTAAT
AGCATTTGTCACCTTCTTTCACTGCTTTGCCATTTTACCTGTGATACTGACTTTCCTGCCACCCTCTaagaaaaa
aaggaaagagaagaaaaaTCCTGAGAACCGGGAGGAAATTGAGTGTGTAGAAATGGTAGATATCGATAGTACCCG
TGTGGTTGACCAAATTACAACAGTG

Exemplary sgRNAs targeting SEQ ID NO: 2 comprise:

SEQ ID NO: 3
TTGTAATTTGGTCAACCACA,
SEQ ID NO: 4
TGTAATTTGGTCAACCACAC,
and
SEQ ID NO: 5
ATGTTCTAGAATACACCAAG

Another exemplary target for CRISPR-mediated modulation of human PTCHD2 comprises SEQ ID NO: 6.

SEQ ID NO: 6
ATGCTGCGGCAGGTTCTGCACAGGGGCTTGAGGACGTGTTTCTCCCGGC
TCGGCCACTTCATTGCCAGTCACCCTGTCTTCTTCGCCTCGGCGCCGGT
GCTCATCTCCATCCTGCTCGGCGCCAGCTTCAGCCGCTACCAGGTCGAG
GAGAGCGTGGAGCACCTGCTGGCGCCCCAGCACAGCCTGGCCAAGATCG
AGCG

Exemplary sgRNAs targeting SEQ ID NO: 6 comprise:

SEQ ID NO: 7
GCTTGAGGACGTGTTTCTCC,
SEQ ID NO: 8
CTACCAGGTCGAGGAGAGCG,
SEQ ID NO: 9
GCGCCGAGGCGAAGAAGACA,
and
SEQ ID NO: 10
CTGCGGCAGGTTCTGCACAG

### Example 10. Suppressing Ptchd1 Using RNA Interference (siRNA or shRNA) or ASOs

RNA interference (including siRNA and shRNA) and ASO agents are designed using methods and tools available in the art for designing such agents. In additions, certain agents, included vectors for expressing shRNA in a cell are commercially available. Such commercially available vectors include, but are not limited to, V3SH7590-228511121 (siRNA5) targeting exon 1, V3SH7590-227764067 (siRNA4) targeting exon 2, and V3SH7590-230688594 (siRNA7) targeting exon 3, each of which is available from Horizon Discovery. These vectors comprise SMARTvector Lentiviral shRNA. The RNAi or ASO agents are administered to cells in a subject using methods available in the art for delivery of such agents.

### Example 11. Use of Pharmacological and Natural Ligands for the Ptch System

Ptchd1 pathway antagonists include agents targeting the Ptch system. Such agents include sonic hedgehog (Shh), which is a natural ligand that blocks Ptch/Ptchd1. Administration of exogenously produced Shh can be administered to a subject to inhibit Ptch1 pathway activity. Agents targeting the Ptch system also include cyclopamine, a pharmacological agent blocking Ptch/Shh interaction, vismodegib, and sonidegib.

1. A method for increasing efficacy of an opioid therapy in a subject comprising administering to the subject one or more Ptched1 antagonists and/or Ptchd1 pathway antagonists.

2. A method for providing or enhancing an analgesic effect mediated by a μ-opioid receptor (MOR) in a subject, comprising administering to the subject an effective amount of one or more Ptched1 antagonists and/or Ptchd1 pathway antagonists.

3. The method of claim 1 or 2, wherein the subject is administered an opioid drug for pain relief.

4. The method of claim 3, wherein the subject is administered the opioid drug prior to, simultaneously with, or subsequent to administration of the one or more Ptched1 antagonists and/or Ptchd1 pathway antagonists.

5. The method of claim 4, wherein the opioid drug is oxycodone, hydrocodone, morphine, codeine, dihydrocodeine, fentanyl, buprenorphine, or methadone.

6. The method of any one of claims 1-5, wherein the subject is a human.

7. A method for suppressing or ameliorating one or more symptoms associated with opioid withdrawal in a subject comprising administering to the subject an effective amount of one or more Ptched1 antagonists and/or Ptchd1 pathway antagonists.

8. The method of claim 7, wherein the subject is suffering from the one or more withdrawal symptoms or is at risk of suffering from the one or more withdrawal symptoms.

9. The method of claim 7 or 8, wherein suppressing or ameliorating one or more symptoms associated with opioid comprises reducing dependence on an opioid drug, assisting the subject in reducing opioid use, or treating an opioid use disorder.

10. The method of any one of claims 7-9, wherein the subject has an opioid use disorder.

11. The method of any one of claims 7-10, wherein the effective amount of the one or more Ptched1 antagonists and/or Ptchd1 pathway antagonists is administered to the subject after discontinuing or reducing use of the opioid drug, or prior to discontinuing or reducing use of the opioid drug.

12. The method of any one of claims 7-11, wherein the opioid drug is selected from the group consisting of: oxycodone, hydrocodone, morphine, codeine, dihydrocodeine, heroin, opium, and fentanyl.

13. The method of any one of claims 7-12, wherein the subject is a human.

14. The method of any of claims 1-13, wherein the Ptched1 antagonists or Ptchd1 pathway antagonist comprises a PTCHD1 RNA interference (RNAi) polynucleotide or a PTCHD1 antisense oligonucleotide (ASO), or a PTCHD1 CRISPR system.

15. The method of any one of claims 1-13, wherein the Ptched1 or Ptchd1 pathway antagonist is selected from the group consisting of: a statin, a cholesterol absorption inhibitor, a PCSK9 inhibitor, a citrate lyase inhibitor, a bile acid sequestrant, a HDL-C raising therapeutic, a fibrate, niacin, an omega-3 fatty acid, an APOE-based therapeutic, or a ABCA1 cholesterol transporter-based therapeutic.

16. The method of claim 15, wherein the Ptched1 or Ptchd1 pathway antagonist is selected from the group consisting of: ezetimibe, alirocumab, evolocumab, bempedoic acid, bempedoic acid-ezetimibe, cholestyramine, colesevelam, colestipol, rosuvastatin, simvastatin, atorvastatin, fenofibrate, gemfibrozil, lovaza, omacor, vascepa, CN-105, CS-6253, a phthalazinone, a pyrazoline, an anti-APOE antibody, an APOE4 ASO, an APOE4 RNAi polynucleotide, bexarotene, probucol, AGB101, epigallocatechin gallate, probucol, a Sonic hedgehog peptide, cyclopamine, vismodegib, and sonidegib.

17. A modified C. elegans animal expressing a mammalian MOR and having a loss of function mutation in the bgg10 gene.

18. The modified C. elegans animal of claim 17, wherein the C. elegans animal further expresses a heterologous hPCTHD1 gene or a heterologous PTR-25 gene.

19. A method of analyzing the effectiveness of a compound in modulating opioid efficacy comprising contacting the modified C. elegans animal of claim 17 or 19 with the compound and monitoring behavior of the animal in response to opioid, wherein decreased movement indicates increased effectiveness of the opioid.


SEQUENCE LISTING (10 sequences)

SEQ ID NO: 1 - DNA, 2664 bp, Homo sapiens
  source 1..2664: mol_type = other DNA
atgctgcggcaggttctgcacaggggcttgaggacgtgtttctcccggctcggccacttcattgccagtcaccctgtcttcttcgcctcggcgccggtgctcatctccatcctgctcggcgccagcttcagccgctaccaggtcgaggagagcgtggagcacctgctggcgccccagcacagcctggccaagatcgagcgcaacctcgttaacagcctcttcccggtcaaccgctccaagcaccgtctctactcggacctgcagacccccgggcgctacggccgggtcatcgtcacctccttccagaaagccaacatgctggaccagcatcacaccgacctgatcttaaagttgcatgctgctgtcaccaagatccaggttccaaggcctggttttaattacacgtttgcccatatatgtatcctgaataatgataagacttgcatcgtggatgacatagtgcacgtcctggaagagctaaagaatgctcgggccaccaatcggaccaattttgctatcacatacccaatcactcacttaaaggacgggagggctgtgtacaatgggcaccagcttgggggcgtcactgtgcacagcaaagaccgggtgaaatctgcagaggccatccagctcacctactacctgcagtcaatcaacagtctcaatgacatggtggctgagaggtgggagtccagcttctgcgacactgtcagactgtttcagaaatccaacagcaaagtcaaaatgtacccttacacgtcctcctcactgagggaagatttccagaagaccagccgcgtatcagaacgttacctggtcaccagcctgattctggtggttaccatggccatcctgtgttgctctatgcaggactgcgtccgcagcaaaccctggctaggcctgctcggattggtgaccataagcctggccactctcactgcagccgggatcatcaatcttactggtgggaaatataattccaccttcctgggagtccctttcgtcatgctaggtcatggattatatgggacttttgaaatgttatcctcctggaggaaaactagagaagaccaacatgttaaagagagaactgcagcagtctatgcagactccatgctctccttttctctcaccactgccatgtacctggtcacctttggcataggggccagccctttcacgaacattgaggcagccaggattttctgctgcaattcctgtattgcaatcttcttcaactacctctatgtactctcgttttatggttccagcctagtgttcactggctacatagaaaacaattaccagcatagtatcttctgtagaaaagtcccaaagcctgaggcattgcaggagaagccggcatggtacaggtttctcctgacggccagattcagtgaggacacagctgaaggcgaggaagcgaacacttacgagagtcacctattggtatgtttcctcaaacgctattactgtgactggataaccaacacctatgtcaagccttttgtagttctcttttaccttatttatatttcctttgccttaatgggctatctgcaggtcagtgaagggtcagaccttagtaacattgtagcaaccgcgacacaaaccattgagtacactactgcccagcaaaagtacttcagcaactacagtcctgtgattgggttttacatatatgagtctatagaatactggaacactagtgtccaagaagatgttctagaatacaccaaggggtttgtgcggatatcctggtttgagagctatttaaattaccttcggaaactcaatgtatccactggcttgcctaagaaaaatttcacagacatgttgaggaattcctttctgaaagcccctcaattttcacattttcaagaggacatcatcttctctaaaaaatacaatgatgaggtcgatgtagtggcctccagaatgtttttggtggccaagaccatggaaacaaacagagaagaactctatgatctcttggaaaccctgaggagactttctgtcacctccaaggtgaagttcatcgtcttcaatccgtcctttgtatacatggatcgatatgcctcctctctgggagcccccctgcacaactcctgcatcagtgctttgttcctgctcttcttctcggcattcctggtggcagattcactgattaacgtctggatcactctcacagttgtgtccgtggagtttggagtgataggtttcatgacattatggaaagtagaactggactgcatttctgtgctatgcttaatttatggaattaattacacaattgacaattgtgctccaatgttatccacatttgttctgggcaaggatttcacaagaactaaatgggtaaaaaatgccctggaagtgcatggggtagctattttacagagttacctctgctatattgttggtctgattcctcttgcagctgtgccttcaaatctgacctgtacactgttcaggtgcttgtttttaatagcatttgtcaccttctttcactgctttgccattttacctgtgatactgactttcctgccaccctctaagaaaaaaaggaaagagaagaaaaatcctgagaaccgggaggaaattgagtgtgtagaaatggtagatatcgatagtacccgtgtggttgaccaaattacaacagtg

SEQ ID NO: 2 - DNA, 1000 bp, synthetic construct
  source 1..1000: mol_type = other DNA
gattgggttttacatatatgagtctatagaatactggaacactagtgtccaagaagatgttctagaatacaccaaggggtttgtgcggatatcctggtttgagagctatttaaattaccttcggaaactcaatgtatccactggcttgcctaagaaaaatttcacagacatgttgaggaattcctttctgaaagcccctcaattttcacattttcaagaggacatcatcttctctaaaaaatacaatgatgaggtcgatgtagtggcctccagaatgtttttggtggccaagaccatggaaacaaacagagaagaactctatgatctcttggaaaccctgaggagactttctgtcacctccaaggtgaagttcatcgtcttcaatccgtcctttgtatacatggatcgatatgcctcctctctgggagcccccctgcacaactcctgcatcagtgctttgttcctgctcttcttctcggcattcctggtggcagattcactgattaacgtctggatcactctcacagttgtgtccgtggagtttggagtgataggtttcatgacattatggaaagtagaactggactgcatttctgtgctatgcttaatttatggaattaattacacaattgacaattgtgctccaatgttatccacatttgttctgggcaaggatttcacaagaactaaatgggtaaaaaatgccctggaagtgcatggggtagctattttacagagttacctctgctatattgttggtctgattcctcttgcagctgtgccttcaaatctgacctgtacactgttcaggtgcttgtttttaatagcatttgtcaccttctttcactgctttgccattttacctgtgatactgactttcctgccaccctctaagaaaaaaaggaaagagaagaaaaatcctgagaaccgggaggaaattgagtgtgtagaaatggtagatatcgatagtacccgtgtggttgaccaaattacaacagtg

SEQ ID NO: 3 - DNA, 20 bp, synthetic construct
  source 1..20: mol_type = other DNA
ttgtaatttggtcaaccaca

SEQ ID NO: 4 - DNA, 20 bp, synthetic construct
  source 1..20: mol_type = other DNA
tgtaatttggtcaaccacac

SEQ ID NO: 5 - DNA, 20 bp, synthetic construct
  source 1..20: mol_type = other DNA
atgttctagaatacaccaag

SEQ ID NO: 6 - DNA, 200 bp, synthetic construct
  source 1..200: mol_type = other DNA
atgctgcggcaggttctgcacaggggcttgaggacgtgtttctcccggctcggccacttcattgccagtcaccctgtcttcttcgcctcggcgccggtgctcatctccatcctgctcggcgccagcttcagccgctaccaggtcgaggagagcgtggagcacctgctggcgccccagcacagcctggccaagatcgagcg

SEQ ID NO: 7 - DNA, 20 bp, synthetic construct
  source 1..20: mol_type = other DNA
gcttgaggacgtgtttctcc

SEQ ID NO: 8 - DNA, 20 bp, synthetic construct
  source 1..20: mol_type = other DNA
ctaccaggtcgaggagagcg

SEQ ID NO: 9 - DNA, 20 bp, synthetic construct
  source 1..20: mol_type = other DNA
gcgccgaggcgaagaagaca

SEQ ID NO: 10 - DNA, 20 bp, synthetic construct
  source 1..20: mol_type = other DNA
ctgcggcaggttctgcacag